(19) United States
(12) Patent Application Publication
(10) Pub. No.: US 2026/0242513 A1
(43) Pub. Date: Aug. 20, 2026

(54) HLA-A3-RESTRICTED T CELL RECEPTORS AGAINST BRAF WITH V600E MUTATION

(71) Applicant: The United States of America, as represented by the Secretary, Department of Health and Human Servic, Bethesda, MD (US)

(72) Inventors: Zhiya Yu, Potomac, MD (US); Catherine M. Ade, Fairfax, VA (US); Matthew J. Sporn, New Rochelle, NY (US); James C. Yang, Bethesda, MD (US); Ken-ichi Hanada, Bethesda, MD (US)

(73) Assignee: The United States of America, as represented by the Secretary, Department of Health and Human Servic, Bethesda, MD (US)

(21) Appl. No.: 19/118,121

(22) PCT Filed: Oct. 30, 2023
(86) PCT No.: PCT/US2023/078156

§ 371 Date: Apr. 3, 2025

Related U.S. Application Data
(60) Provisional application No. 63/381,587, filed on Oct. 31, 2022.

(51) Int. Cl. C07K 16/40; A61K 38/00; A61P 35/00; C07K 14/705; G01N 33/575

(52) U.S. Cl. CPC C07K 16/40; A61P 35/00; C07K 14/7051; G01N 33/5758; A61K 38/00; C07K 2317/31; G01N 2333/7051

(57) ABSTRACT

Disclosed is an isolated or purified T cell receptor (TCR), wherein the TCR has antigenic specificity for a human BRAF^{V600E} amino acid sequence. Related polypeptides and proteins, as well as related nucleic acids, recombinant expression vectors, host cells, populations of cells, and pharmaceutical compositions are also provided. Also disclosed are methods of detecting the presence of cancer in a mammal and methods of treating or preventing cancer in a mammal.

### CROSS-REFERENCE TO RELATED APPLICATION

This patent application claims the benefit of U.S. Provisional Patent Application No. 63/381,587, filed Oct. 31, 2022, which is incorporated by reference in its entirety herein.

### STATEMENT REGARDING FEDERALLY SPONSORED RESEARCH OR DEVELOPMENT

This invention was made with Government support under project number ZIA BC 011843 by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 60,248 Byte XML file named “768484.xml,” dated Oct. 27, 2023.

### BACKGROUND OF THE INVENTION

Some cancers may have very limited treatment options, particularly when the cancer becomes metastatic and unresectable. Despite advances in treatments such as, for example, surgery, chemotherapy, and radiation therapy, the prognosis for many cancers, such as, for example, melanoma, hairy cell leukemia, non-Hodgkin lymphoma, thyroid cancer, ovarian cancer, lung adenocarcinoma, colorectal cancer, and brain cancer, may be poor. Accordingly, there exists an unmet need for additional treatments for cancer.

### BRIEF SUMMARY OF THE INVENTION

An aspect of the invention provides an isolated or purified T cell receptor (TCR) having antigenic specificity for a human BRAF^{V600E} amino acid sequence, wherein the TCR comprises the amino acid sequences of: (a) all of SEQ ID NO: 1-3; (b) all of SEQ ID NO: 4-6; (c) all of SEQ ID NO: 1-6; (d) all of SEQ ID NO: 11-13; (e) all of SEQ ID NO: 14-16; or (f) all of SEQ ID NO: 11-16.

Another aspect of the invention provides an isolated or purified polypeptide comprising a functional portion of the inventive TCR, wherein the functional portion comprises the amino acid sequences of: (a) all of SEQ ID NO: 1-3; (b) all of SEQ ID NO: 4-6; (c) all of SEQ ID NO: 1-6; (d) all of SEQ ID NO: 11-13; (e) all of SEQ ID NO: 14-16; or (f) all of SEQ ID NO: 11-16.

Still another aspect of the invention provides an isolated or purified protein comprising at least one of the inventive polypeptides.

Another aspect of the invention provides a bispecific engager TCR fusion protein comprising (i) any of the inventive TCRs, polypeptides, or proteins described herein and (ii) an anti-CD3 engager.

Aspects of the invention further provide nucleic acids, recombinant expression vectors, host cells, populations of cells, and pharmaceutical compositions relating to the inventive TCRs, polypeptides, and proteins.

Another aspect of the invention provides an isolated or purified nucleic acid comprising, from 5′ to 3′, a first nucleic acid sequence and a second nucleotide sequence, wherein the first and second nucleotide sequence, respectively, encode the amino sequences of SEQ ID NOs: 7 and 8; 8 and 7; 9 and 10; 10 and 9; 17 and 18; 18 and 17; 19 and 20; 20 and 19; 27 and 28; 28 and 27; 29 and 30; 30 and 29; 31 and 32; 32 and 31; 33 and 34; 34 and 33; 35 and 36; 36 and 35; 37 and 38; 38 and 37; 39 and 40; 40 and 39; 41 and 42; or 42 and 41.

Methods of detecting the presence of cancer in a mammal, methods of treating or preventing cancer in a mammal, methods of inducing an immune response against a cancer in a mammal, methods of producing a host cell expressing a TCR that has antigenic specificity for the peptide of KIGDFGLATEK (SEQ ID NO: 45), and methods of producing the inventive TCRs, polypeptides, and proteins, are further provided by aspects of the invention.

### BRIEF DESCRIPTION OF THE SEVERAL VIEWS OF THE DRAWING(S)

FIGS. 1A-1B are graphs showing the concentration of IFN-gamma (pg/mL) secreted following co-culture of target cells with effector cells. HLA-A*03:01+ target cells were pulsed with the V600E BRAF peptide KIGDFGLATEK (mutated) (SEQ ID NO: 45) (1A) or KIGDFGLATVK (wild-type) (SEQ ID NO: 46) (1B) at the peptide concentrations shown. Effector cells were T cells from healthy human donors transduced with a retroviral vector encoding mTCR 4734_1 (circles) or mTCR 5489_4 (triangles). T cells transduced with an empty vector (mock) (squares) served as a control.

FIG. 2 is a graph showing the concentration of IFN-gamma (pg/mL) secreted following co-culture of target cells with effector cells. Target cells were an HLA-A*03:01+ positive tumor cell line K562 that was either untransfected (unshaded bars) or transfected with V600E BRAF (shaded bars). Effector cells were T cells from healthy human donors transduced with a retroviral vector encoding mTCR 4734_1 or mTCR 5489_4. T cells transduced with an empty vector (mock) served as a control.

FIGS. 3A-3B are graphs showing the average cell area per image (μm²) measured (with INCUCYTE live cell analysis system, Sartorius Corporation, Bohemia, NY) over a 48-hour co-culture of target cells with effector cells in media with (3B) and without (3A) HLA-blocking antibody, W6/32. Target cells were an HLA-A*03:01+ positive tumor cell line mel 3733. Effector cells were T cells from healthy human donors transduced with a retroviral vector encoding mTCR 4734_1 (circles) or mTCR 5489_4 (triangles). T cells transduced with an empty vector (mock) (squares) served as a control.

### DETAILED DESCRIPTION OF THE INVENTION

The BRAF protein belongs to the RAF family of serine/threonine protein kinases. BRAF is also referred to as “B-Raf Proto-Oncogene, Serine/Threonine Kinase,” “BRAF1” or “V-Raf Murine Sarcoma Viral Oncogene Homolog B.” BRAF is involved in regulating the mitogen-activated protein (MAP) kinase/extracellular signal-regulated kinase (ERK) signaling pathway, which affects cell division, differentiation, and secretion. V600E BRAF mutations are spontaneous changes in the BRAF gene that cause cells to continuously divide. This mutation can be found in any of a variety of human cancers such as, for example, melanoma, hairy cell leukemia, non-Hodgkin lymphoma, thyroid cancer, ovarian cancer, lung adenocarcinoma, colorectal cancer, and brain cancer. WT (normal) full-length BRAF (isoform 1) comprises the amino acid sequence of SEQ ID NO: 48.

An aspect of the invention provides an isolated or purified TCR having antigenic specificity for a human BRAF^{V600E} amino acid sequence. Hereinafter, references to a “TCR” also refer to functional portions and functional variants of the TCR, unless specified otherwise. Mutations of BRAF are defined herein by reference to the amino acid sequence of full-length, WT BRAF (SEQ ID NO: 48). Mutations of BRAF are described herein by reference to the amino acid residue present at a particular position, followed by the position number, followed by the amino acid with which that residue has been replaced in the particular mutation under discussion. A BRAF amino acid sequence (e.g., a BRAF peptide) may comprise fewer than all of the amino acid residues of the full-length, WT BRAF protein. Accordingly, the position numbers are defined herein by reference to the WT full-length BRAF protein (namely, SEQ ID NO: 48) with the understanding that the actual position of the corresponding residue in a particular example of a BRAF amino acid sequence may be different. Because the positions are as defined by SEQ ID NO: 48, the term “V600” refers to the valine present at position 600 of SEQ ID NO: 48, and “V600E” indicates that the valine present at position 600 of SEQ ID NO: 48 is replaced by glutamic acid. For example, when a particular example of a BRAF amino acid sequence is, e.g., KIGDFGLATVK (SEQ ID NO: 46) (an exemplary WT BRAF peptide corresponding to contiguous amino acid residues 591 to 601 of SEQ ID NO: 48), “V600E” refers to a substitution of the underlined valine in SEQ ID NO: 46 with glutamic acid, even though the actual position of the underlined valine in SEQ ID NO: 46 is 10. Human BRAF amino acid sequences with the V600E mutation are hereinafter referred to as “V600E BRAF” or “BRAF^{V600E}.”

Human BRAF has 13 isoforms. The BRAF mutations described herein are conserved over all 13 BRAF isoforms. Accordingly, the inventive TCRs may have antigenic specificity for the mutated BRAF amino acid sequence described herein found in any of the 13 BRAF isoforms. Because the positions are as defined by SEQ ID NO: 48, then the actual positions of the amino acid sequence of a particular isoform of BRAF are defined relative to the corresponding positions of SEQ ID NO: 48, and the positions as defined by SEQ ID NO: 48 may be different than the actual positions in a particular isoform. Thus, for example, mutations refer to a replacement of an amino acid residue in the amino acid sequence of a particular isoform of BRAF corresponding to the indicated position of the 766-amino acid sequence of SEQ ID NO: 48 with the understanding that the actual positions in the isoform may be different.

In an aspect of the invention, the TCR has antigenic specificity for a human BRAF amino acid sequence with a substitution of valine at position 600 with glutamic acid. In this regard, aspects of the invention provide TCRs with antigenic specificity for any human BRAF protein, polypeptide or peptide amino acid sequence with the V600E mutation.

In an aspect of the invention, the TCRs have antigenic specificity for a BRAF peptide with the V600E mutation described above, wherein the mutated BRAF peptide has any length suitable for binding to any of the HLA-A3 molecules described herein. For example, the TCRs may have antigenic specificity for a BRAF peptide with the V600E mutation, the BRAF peptide having a length of about 9 to about 11 amino acid residues. The TCRs may have antigenic specificity for a mutated BRAF peptide comprising contiguous amino acid residues of mutated BRAF protein which include the V600E mutation. In an aspect of the invention, the TCRs may have antigenic specificity for a BRAF peptide with the V600E mutation, the mutated BRAF peptide having a length of about 9 amino acid residues, about 10 amino acid residues, or about 11 amino acid residues. An example of a specific peptide with the V600E mutation, which may be recognized by the inventive V600E TCRs, is the 11-mer peptide KIGDFGLATEK (SEQ ID NO: 45). In an aspect of the invention, the TCR does not have antigenic specificity for the wild-type human BRAF amino acid sequence of KIGDFGLATVK (SEQ ID NO: 46).

In an aspect of the invention, the inventive TCRs are able to recognize mutated BRAF presented by an HLA-A3 molecule. In this regard, the TCRs may elicit an immune response upon binding to mutated BRAF presented by an HLA-A3 molecule. The inventive TCRs may bind to the HLA-A3 molecule in addition to mutated BRAF. The HLA-A3 molecule is a heterodimer of an α chain and β2 microglobulin. The HLA-A3 α chain may be encoded by an HLA-A3 gene. β2 microglobulin binds non-covalently to the alpha1, alpha2 and alpha3 domains of the alpha chain to build the HLA-A3 complex. The HLA-A3 molecule may be any HLA-A3 molecule. Examples of HLA-A3 molecules may include, but are not limited to, HLA-A*3:01, HLA-A*3:02, or HLA-A*3:05.

The TCRs of the invention may provide any one or more of a variety of advantages, including when expressed by cells used for adoptive cell transfer. V600E BRAF is expressed by cancer cells and is not expressed by normal, noncancerous cells. Without being bound to a particular theory or mechanism, it is believed that the inventive TCRs advantageously target the destruction of cancer cells while minimizing or eliminating the destruction of normal, non-cancerous cells, thereby reducing, for example, by minimizing or eliminating, toxicity. The inventive TCRs may, advantageously, successfully treat or prevent V600E BRAF-positive cancers that do not respond to other types of treatment such as, for example, chemotherapy, surgery, or radiation. Additionally, the inventive TCRs may provide highly avid recognition of V600E BRAF, which may provide the ability to recognize unmanipulated tumor cells (e.g., tumor cells that have not been treated with interferon (IFN)-γ, transfected with a vector encoding one or both of V600E BRAF and HLA-A3, pulsed with a V600E BRAF peptide, or a combination thereof). Moreover, the frequency of the HLA-A3 allele in the total U.S. population is about 9% to about 11% such that about 20% of the Caucasian population in the United States will have the HLA-A3 gene (twice the allele frequency). Accordingly, the inventive TCRs may increase the number of immunotherapy-eligible cancer patients to include those patients that express HLA-A3 who may not be eligible for immunotherapy using TCRs that recognize mutated BRAF presented by other MHC molecules.

The phrase “antigenic specificity,” as used herein, means that the TCR can specifically bind to and immunologically recognize V600E BRAF with high avidity. For example, a TCR may be considered to have “antigenic specificity” for V600E BRAF if about 1×10⁴ to about 1×10⁵ T cells expressing the TCR secrete at least about 200 pg/mL or more (e.g., 200 pg/mL or more, 300 pg/mL or more, 400 pg/mL or more, 500 pg/mL or more, 600 pg/mL or more, 700 pg/mL or more, 1000 pg/mL or more, 5,000 pg/mL or more, 7,000 pg/mL or more, 10,000 pg/mL or more, 20,000 pg/mL or more, or a range defined by any two of the foregoing values) of IFN-γ upon co-culture with antigen-negative, HLA-A3 molecule positive target cells pulsed with a low concentration of V600E BRAF peptide (e.g., about 1000 ng/mL or less, 100 ng/ml or less, 10 ng/ml or less, or 1 ng/ml or less, or a range defined by any two of the foregoing values) while co-culture with the antigen-negative, HLA-A3 molecule positive target cells pulsed with a similar concentration of the unmutated wild type peptide results in IFN-gamma secretion of less than half of that amount. In some aspects, the differential is large with IFN-gamma secretion upon co-culture with the target cell pulsed with the wild type peptide being negligible (less than 100 pg/mL), while the IFN-gamma secretion upon co-culture with the target cell pulsed with the mutated V600E peptide may be 10 fold higher (or greater). IFN-γ secretion may be measured by methods known in the art such as, for example, enzyme-linked immunosorbent assay (ELISA).

Alternatively or additionally, a TCR may be considered to have “antigenic specificity” for V600E BRAF if T cells expressing the TCR secrete at least twice (e.g., ten times) as much IFN-γ upon co-culture with antigen-negative, HLA-A3 molecule positive target cells into which a nucleotide sequence encoding V600E BRAF has been introduced such that the target cell expresses V600E BRAF as compared to the amount of IFN-γ secretion induced by co-culture with a negative control. The negative control may be, for example, (i) T cells expressing the TCR, co-cultured with (a) the unmodified antigen-negative, HLA-A3 molecule positive target cells or (b) the antigen-negative, HLA-A3 molecule positive target cells into which a nucleotide sequence encoding the corresponding sequence of wild type BRAF has been introduced. IFN-γ secretion may be measured by methods known in the art such as, for example, enzyme-linked immunosorbent assay (ELISA).

Alternatively or additionally, a TCR may be considered to have “antigenic specificity” for V600E BRAF if at least twice (e.g., five times) as many of the numbers of T cells expressing the TCR secrete IFN-γ upon co-culture with (a) antigen-negative, HLA-A3 molecule positive target cells pulsed with a low concentration of V600E BRAF peptide or (b) antigen-negative, HLA-A3 molecule positive target cells into which a nucleotide sequence encoding V600E BRAF has been introduced such that the target cell expresses V600E BRAF as compared to the numbers of negative control T cells that secrete IFN-γ. The HLA-A3 molecule, concentration of peptide, and the negative control may be as described herein with respect to other aspects of the invention. The numbers of cells secreting IFN-γ may be measured by methods known in the art such as, for example, ELISPOT.

Alternatively or additionally, a TCR may be considered to have “antigenic specificity” for V600E BRAF if T cells expressing the TCR upregulate expression of one or more T-cell activation markers as measured by, for example, flow cytometry after stimulation with target cells expressing V600E BRAF. Examples of T-cell activation markers include 4-1BB, OX40, CD107a, CD69, and cytokines that are upregulated upon antigen stimulation (e.g., tumor necrosis factor (TNF), interleukin (IL)-2, etc.).

An aspect of the invention provides a TCR comprising two polypeptides (i.e., polypeptide chains), such as an alpha (α) chain of a TCR, a beta (β) chain of a TCR, a gamma (γ) chain of a TCR, a delta (δ) chain of a TCR, or a combination thereof. The polypeptides of the inventive TCR can comprise any amino acid sequence, provided that the TCR has antigenic specificity for V600E BRAF. In some aspects, the TCR is non-naturally occurring.

In an aspect of the invention, the TCR comprises two polypeptide chains, each of which comprises a variable region comprising a complementarity determining region (CDR) 1, a CDR2, and a CDR3 of a TCR. In an aspect of the invention, the TCR comprises a first polypeptide chain comprising a CDR1 comprising the amino acid sequence of SEQ ID NO: 1 (CDR1 of α chain of mTCR 4734_1), a CDR2 comprising the amino acid sequence of SEQ ID NO: 2 (CDR2 of α chain of mTCR 4734_1), and a CDR3 comprising the amino acid sequence of SEQ ID NO: 3 (CDR3 of α chain of mTCR 4734_1), and a second polypeptide chain comprising a CDR1 comprising the amino acid sequence of SEQ ID NO: 4 (CDR1 of β chain of mTCR 4734_1), a CDR2 comprising the amino acid sequence of SEQ ID NO: 5 (CDR2 of β chain of mTCR 4734_1), and a CDR3 comprising the amino acid sequence of SEQ ID NO: 6 (CDR3 of β chain of mTCR 4734_1). In this regard, the inventive TCR can comprise any one or more of the amino acid sequences selected from the group consisting of SEQ ID NOs: 1-6. In an aspect of the invention, the TCR comprises the amino acid sequences of: (a) all of SEQ ID NOs: 1-3, (b) all of SEQ ID NOs: 4-6, or (c) all of SEQ ID NOs: 1-6. In an especially preferred aspect, the TCR comprises the amino acid sequences of all of SEQ ID NOs: 1-6.

In an aspect of the invention, the TCR comprises a first polypeptide chain comprising a CDR1 comprising the amino acid sequence of SEQ ID NO: 11 (CDR1 of a chain of mTCR 5489_4), a CDR2 comprising the amino acid sequence of SEQ ID NO: 12 (CDR2 of α chain of mTCR 5489_4), and a CDR3 comprising the amino acid sequence of SEQ ID NO: 13 (CDR3 of α chain of mTCR 5489_4), and a second polypeptide chain comprising a CDR1 comprising the amino acid sequence of SEQ ID NO: 14 (CDR1 of β chain of mTCR 5489_4), a CDR2 comprising the amino acid sequence of SEQ ID NO: 15 (CDR2 of β chain of mTCR 5489_4), and a CDR3 comprising the amino acid sequence of SEQ ID NO: 16 (CDR3 of β chain of mTCR 5489_4). In this regard, the inventive TCR can comprise any one or more of the amino acid sequences selected from the group consisting of SEQ ID NOs: 11-16. In an aspect of the invention, the TCR comprises the amino acid sequences of: (a) all of SEQ ID NOs: 11-13, (b) all of SEQ ID NOs: 14-16, or (c) all of SEQ ID NOs: 11-16. In an especially preferred aspect, the TCR comprises the amino acid sequences of all of SEQ ID NOs: 11-16.

In an aspect of the invention, the TCR comprises an amino acid sequence of a variable region of a TCR comprising the CDRs set forth above. In this regard, the TCR can comprise the amino acid sequence of: (i) SEQ ID NO: 7 (predicted sequence of variable region of mTCR 4734_1 α chain without N-terminal signal peptide); (ii) SEQ ID NO: 8 (predicted sequence of variable region of mTCR 4734_1 β chain without N-terminal signal peptide); (iii) SEQ ID NO: 9 (variable region of mTCR 4734_1 α chain with N-terminal signal peptide); (iv) SEQ ID NO: 10 (variable region of mTCR 4734_1 β chain with N-terminal signal peptide); (v) both of SEQ ID NOs: 7 and 8; or (vi) both of SEQ ID NOs: 9 and 10.

In an aspect of the invention, the TCR comprises the amino acid sequence of: (i) SEQ ID NO: 17 (predicted sequence of variable region of mTCR 5489_4 α chain without N-terminal signal peptide); (ii) SEQ ID NO: 18 (predicted sequence of variable region of mTCR 5489_4 β chain without N-terminal signal peptide); (iii) SEQ ID NO: 19 (variable region of mTCR 5489_4 α chain with N-terminal signal peptide); (iv) SEQ ID NO: 20 (variable region of mTCR 5489_4 β chain with N-terminal signal peptide); (v) both of SEQ ID NOs: 17 and 18; or (vi) both of SEQ ID NOs: 19 and 20.

The inventive TCRs may further comprise an α chain constant region and a β chain constant region. The constant region may be derived from any suitable species such as, e.g., human or mouse. In an aspect of the invention, the TCRs further comprise murine α and β chain constant regions or human α and β chain constant regions. As used herein, the term “murine” or “human,” when referring to a TCR or any component of a TCR described herein (e.g., CDR, variable region, constant region, α chain, and/or β chain), means a TCR (or component thereof) which is derived from a mouse or a human, respectively, i.e., a TCR (or component thereof) that originated from or was, at one time, expressed by a mouse T cell or a human T cell, respectively.

An aspect of the invention provides a murine TCR comprising a murine variable region and a murine constant region, wherein the TCR has antigenic specificity for a mutated human BRAF amino acid sequence presented by an HLA-A3 molecule. The murine constant region may provide any one or more advantages. For example, the murine constant region may diminish mispairing of the inventive TCR with the endogenous TCRs of the host cell into which the inventive TCR is introduced when the host cell is not a murine host cell, e.g., a human host cell. Alternatively or additionally, the murine constant region may increase expression of the inventive TCR as compared to the same TCR with a human constant region. The TCR may comprise the amino acid sequence of SEQ ID NO: 24 (wild-type (WT) murine α chain constant region), SEQ ID NO: 25 (WT murine mTCR 5489_4 β chain constant region), SEQ ID NO: 26 (WT murine mTCR 4734_1 β chain constant region), both SEQ ID NOs: 24 and 25, or both SEQ ID NOs: 24 and 26. Preferably, the inventive TCR comprises the amino acid sequences of both of SEQ ID NOs: 24 and 25 or both of SEQ ID NOs: 24 and 26.

The TCR may comprise any of the murine constant regions described herein in combination with any of the CDR regions as described herein with respect to other aspects of the invention. In this regard, the TCR may comprise the amino acid sequences of: (a) all of SEQ ID NOs: 1-3 and 24; (b) all of SEQ ID NOs: 4-6 and 25; (c) all of SEQ ID NOs: 1-6 and 24-25; (d) all of SEQ ID NOs: 4-6 and 26; (e) all of SEQ ID NOs: 1-6, 24, and 26; (f) all of SEQ ID NOs: 11-13 and 24; (g) all of SEQ ID NOs: 14-16 and 25; (h) all of SEQ ID NOs: 11-16, 24, and 25; (i) all of SEQ ID NOs: 14-16 and 26; or (j) all of SEQ ID NOs: 11-16, 24, and 26.

In another aspect of the invention, the TCR may comprise any of the murine constant regions described herein in combination with any of the variable regions described herein with respect to other aspects of the invention. In this regard, the TCR may comprise the amino acid sequences of: (i) both of SEQ ID NOs: 7 and 24; (ii) both of SEQ ID NOs: 8 and 25; (iii) both of SEQ ID NOs: 8 and 26; (iv) both of SEQ ID NOs: 9 and 24; (v) both of SEQ ID NOs: 10 and 25; (vi) both of SEQ ID NOs: 10 and 26; (vii) all of SEQ ID NOs: 7-8 and 24-25; (viii) all of SEQ ID NOs: 9-10 and 24-25; (ix) all of SEQ ID NOs: 7-8, 24, and 26; or (x) all of SEQ ID NOs: 9-10, 24, and 26.

In an aspect of the invention, the TCR comprises a substituted constant region. In this regard, the TCR may comprise the amino acid sequence of any of the TCRs described herein with one, two, three, or four amino acid substitution(s) in the constant region of one or both of the α and β chain. Preferably, the TCR comprises a murine constant region with one, two, three, or four amino acid substitution(s) in the murine constant region of one or both of the α and β chains. In an especially preferred aspect, the TCR comprises a murine constant region with one, two, three, or four amino acid substitution(s) in the murine constant region of the α chain and one amino acid substitution in the murine constant region of the β chain. In some aspects, the TCRs comprising the substituted constant region may provide one or more of increased recognition of V600E BRAF⁺ targets, increased expression by a host cell, diminished mispairing with endogenous TCRs, and increased anti-tumor activity as compared to the parent TCR comprising an unsubstituted (wild-type) constant region. In general, the substituted amino acid sequences of the murine constant regions of the TCR α and β chains, SEQ ID NOs: 21 and 22, respectively, correspond with all or portions of the unsubstituted murine constant region amino acid sequences SEQ ID NOs: 24 and 25, respectively, with SEQ ID NO: 21 having one, two, three, or four amino acid substitution(s) when compared to SEQ ID NO: 24 and SEQ ID NO: 22 having one amino acid substitution when compared to SEQ ID NO: 25. The substituted amino acid sequence of the murine constant region of the TCR β chain, SEQ ID NO: 23, corresponds with all or portions of the unsubstituted murine constant region amino acid sequence SEQ ID NO: 26, with SEQ ID NO: 23 having one amino acid substitution when compared to SEQ ID NO: 26.

In this regard, an aspect of the invention provides a TCR comprising the amino acid sequences of (a) an α chain constant region comprising the amino acid sequence of SEQ ID NO: 21, wherein: (i) X at position 47 of SEQ ID NO: 21 is Thr or Cys; (ii) X at position 111 of SEQ ID NO: 21 is Ser, Ala, Val, Leu, Ile, Pro, Phe, Met, or Trp; (iii) X at position 113 of SEQ ID NO: 21 is Met, Ala, Val, Leu, Ile, Pro, Phe, or Trp; and (iv) X at position 114 of SEQ ID NO: 21 is Gly, Ala, Val, Leu, Ile, Pro, Phe, Met, or Trp; (b) a β chain constant region comprising the amino acid sequence of SEQ ID NO: 22, wherein X at position 59 of SEQ ID NO: 22 is Ser or Cys; (c) a β chain constant region comprising the amino acid sequence of SEQ ID NO: 23, wherein X at position 59 of SEQ ID NO: 23 is Ser or Cys; (d) both SEQ ID NOs: 21 and 22; or (e) both SEQ ID NOs: 21 and 23. In an aspect of the invention, the TCR comprising SEQ ID NO: 21 does not comprise SEQ ID NO: 24 (unsubstituted murine constant region of α chain). In an aspect of the invention, the TCR comprising SEQ ID NO: 22 does not comprise SEQ ID NO: 25 (unsubstituted murine constant region of β chain). In an aspect of the invention, the TCR comprising SEQ ID NO: 23 does not comprise SEQ ID NO: 26 (unsubstituted murine constant region of β chain).

In an aspect of the invention, the TCR comprises an α chain comprising a variable region and a constant region and a β chain comprising a variable region and a constant region. In this regard, the TCR may comprise: (a) an α chain comprising the amino acid sequence of SEQ ID NO: 27 (mTCR 4734_1 α chain with N-terminal signal peptide), wherein: (i) X at position 177 of SEQ ID NO: 27 is Thr or Cys; (ii) X at position 241 of SEQ ID NO: 27 is Ser, Ala, Val, Leu, Ile, Pro, Phe, Met, or Trp; (iii) X at position 243 of SEQ ID NO: 27 is Met, Ala, Val, Leu, Ile, Pro, Phe, or Trp; and (iv) X at position 244 of SEQ ID NO: 27 is Gly, Ala, Val, Leu, Ile, Pro, Phe, Met, or Trp; (b) a β chain comprising the amino acid sequence of SEQ ID NO: 28 (mTCR 4734_1 β chain with N-terminal signal peptide), wherein X at position 191 of SEQ ID NO: 28 is Ser or Cys; (c) both SEQ ID NOs: 27 and 28; (d) an α chain comprising the amino acid sequence of SEQ ID NO: 29 (mTCR 4734_1 α chain predicted sequence without N-terminal signal peptide), wherein: (i) X at position 157 of SEQ ID NO: 29 is Thr or Cys; (ii) X at position 221 of SEQ ID NO: 29 is Ser, Ala, Val, Leu, Ile, Pro, Phe, Met, or Trp; (iii) X at position 223 of SEQ ID NO: 29 is Met, Ala, Val, Leu, Ile, Pro, Phe, or Trp; and (iv) X at position 224 of SEQ ID NO: 29 is Gly, Ala, Val, Leu, Ile, Pro, Phe, Met, or Trp; (e) a β chain comprising the amino acid sequence of SEQ ID NO: 30 (mTCR 4734_1 β chain predicted sequence without N-terminal signal peptide), wherein X at position 172 of SEQ ID NO: 30 is Ser or Cys; (f) both SEQ ID NOs: 29 and 30; (g) SEQ ID NO: 31 (mTCR 4734_1 α chain with N-terminal signal peptide); (h) SEQ ID NO: 32 (mTCR 4734_1 β chain with N-terminal signal peptide); (i) SEQ ID NO: 33 (mTCR 4734_1 α chain predicted sequence without N-terminal signal peptide); (j) SEQ ID NO: 34 (mTCR 4734_1 β chain predicted sequence without N-terminal signal peptide); (k) both SEQ ID NOs: 31 and 32; (1) both SEQ ID NO: 33 and 34; (m) an α chain comprising the amino acid sequence of SEQ ID NO: 35 (mTCR 5489_4 α chain with N-terminal signal peptide), wherein: (i) X at position 184 of SEQ ID NO: 35 is Thr or Cys; (ii) X at position 248 of SEQ ID NO: 35 is Ser, Ala, Val, Leu, Ile, Pro, Phe, Met, or Trp; (iii) X at position 250 of SEQ ID NO: 35 is Met, Ala, Val, Leu, Ile, Pro, Phe, or Trp; and (iv) X at position 251 of SEQ ID NO: 35 is Gly, Ala, Val, Leu, Ile, Pro, Phe, Met, or Trp; (n) a β chain comprising the amino acid sequence of SEQ ID NO: 36 (mTCR 5489_4 β chain with N-terminal signal peptide), wherein X at position 188 of SEQ ID NO: 36 is Ser or Cys; (o) both SEQ ID NOs: 35 and 36; (p) an α chain comprising the amino acid sequence of SEQ ID NO: 37 (mTCR 5489_4 α chain predicted sequence without N-terminal signal peptide), wherein: (i) X at position 163 of SEQ ID NO: 37 is Thr or Cys; (ii) X at position 227 of SEQ ID NO: 37 is Ser, Ala, Val, Leu, Ile, Pro, Phe, Met, or Trp; (iii) X at position 229 of SEQ ID NO: 37 is Met, Ala, Val, Leu, Ile, Pro, Phe, or Trp; and (iv) X at position 230 of SEQ ID NO: 37 is Gly, Ala, Val, Leu, Ile, Pro, Phe, Met, or Trp; (q) a β chain comprising the amino acid sequence of SEQ ID NO: 38 (mTCR 5489_4 β chain predicted sequence without N-terminal signal peptide), wherein X at position 169 of SEQ ID NO: 38 is Ser or Cys; (r) both SEQ ID NOs: 37 and 38; (s) SEQ ID NO: 39 (mTCR 5489_4 α chain with N-terminal signal peptide); (t) SEQ ID NO: 40 (mTCR 5489_4 β chain with N-terminal signal peptide); (u) SEQ ID NO: 41 (mTCR 5489_4 α chain predicted sequence without N-terminal signal peptide); (v) SEQ ID NO: 42 (mTCR 5489_4 β chain predicted sequence without N-terminal signal peptide); (w) both SEQ ID NOs: 39 and 40; or (x) both SEQ ID NOs: 41 and 42.

In an aspect of the invention, the substituted constant region includes cysteine substitutions in the constant region of one or both of the α and β chains to provide a cysteine-substituted TCR. Opposing cysteines in the α and the β chains provide a disulfide bond that links the constant regions of the α and the β chains of the substituted TCR to one another and which is not present in a TCR comprising the unsubstituted murine constant regions. In this regard, the TCR may be a cysteine-substituted TCR in which one or both of (i) the native Thr at position 47 (Thr47) of SEQ ID NO: 24 and (ii) the native Ser at position 59 (Ser59) of SEQ ID NO: 25 or SEQ ID NO: 26 may be substituted with Cys. Preferably, both of the native Thr47 of SEQ ID NO: 24 and the native Ser59 of SEQ ID NO: 25 are substituted with Cys or both of the native Thr47 of SEQ ID NO: 24 and the native Ser59 of SEQ ID NO: 26 are substituted with Cys. Examples of cysteine-substituted TCR constant regions sequences are set forth in Table 1. In an aspect of the invention, the cysteine-substituted TCR comprises (i) SEQ ID NO: 21, (ii) SEQ ID NO: 22, (iii) SEQ ID NO: 23, (iv) both of SEQ ID NOs: 21 and 22, or (v) both of SEQ ID NOs: 21 and 23, wherein both of SEQ ID NOs: 21 and 22 or both of SEQ ID NOs: 21 and 23 are as defined in Table 1. The cysteine-substituted TCRs of the invention may include the substituted constant region in addition to any of the CDRs or variable regions described herein.

In an aspect of the invention, the cysteine-substituted TCR comprises a full length α chain and a full-length β chain. Examples of cysteine-substituted TCR α chain and β chain sequences are set forth in Table 1. In an aspect of the invention, the TCR comprises: (i) SEQ ID NO: 27, (ii) SEQ ID NO: 28, (iii) SEQ ID NO: 29, (iv) SEQ ID NO: 30, (v) SEQ ID NO: 35, (vi) SEQ ID NO: 36, (vii) SEQ ID NO: 37, (viii) SEQ ID NO: 38, (ix) both of SEQ ID NOs: 27 and 28, (x) both of SEQ ID NOs: 29 and 30, (xi) both of SEQ ID NOs: 35 and 36, (xii) both of SEQ ID NOs: 37 and 38, wherein all of SEQ ID NOs: 27-30 and SEQ ID NOS: 35-38 are as defined in Table 1.

TABLE 1

| SEQ ID NO: | Definitions of “X” |
|---|---|
| SEQ ID NO: 21 | X at position 47 is Cys, |
| (constant region α chain) | X at position 111 is Ser, |
| | X at position 113 is Met, and |
| | X at position 114 is Gly. |
| SEQ ID NO: 22 | X at position 59 is Cys |
| (constant region β chain) | |
| SEQ ID NO: 23 | X at position 59 is Cys |
| (constant region β chain) | |
| SEQ ID NO: 27 | X at position 177 is Cys, |
| (mTCR 4734_1 α chain with N-terminal | X at position 241 is Ser, |
| signal peptide) | X at position 243 is Met, and |
| | X at position 244 is Gly. |
| SEQ ID NO: 28 | X at position 191 is Cys |
| (mTCR 4734_1 β chain with N-terminal | |
| signal peptide) | |
| SEQ ID NO: 29 | X at position 157 is Cys, |
| (mTCR 4734_1 α chain predicted | X at position 221 is Ser, |
| sequence without N-terminal signal | X at position 223 is Met, and |
| peptide) | X at position 224 is Gly. |
| SEQ ID NO: 30 | X at position 172 is Cys |
| (mTCR 4734_1 β chain predicted | |
| sequence without N-terminal signal | |
| peptide) | |
| SEQ ID NO: 35 | X at position 184 is Cys, |
| (mTCR 5489_4 α chain with N-terminal | X at position 248 is Ser, |
| signal peptide) | X at position 250 is Met, and |
| | X at position 251 is Gly. |
| SEQ ID NO: 36 | X at position 188 is Cys |
| (mTCR 5489_4 β chain with N-terminal | |
| signal peptide) | |
| SEQ ID NO: 37 | X at position 163 is Cys, |
| (mTCR 5489_4 α chain predicted | X at position 227 is Ser, |
| sequence without N-terminal signal | X at position 229 is Met, and |
| peptide) | X at position 230 is Gly. |
| SEQ ID NO: 38 | X at position 169 is Cys |
| (mTCR 5489_4 β chain predicted | |
| sequence without N-terminal | |
| signal peptide) | |

In an aspect of the invention, the substituted amino acid sequence includes substitutions of one, two, or three amino acids in the transmembrane (TM) domain of the constant region of the α chain with a hydrophobic amino acid to provide a hydrophobic amino acid-substituted TCR (also referred to herein as an “LVL-modified TCR”). The hydrophobic amino acid substitution(s) in the TM domain of the TCR may increase the hydrophobicity of the TM domain of the TCR as compared to a TCR that lacks the hydrophobic amino acid substitution(s) in the TM domain. In this regard, the TCR is an LVL-modified TCR in which one, two, or three of the native Ser111, Met113, and Gly114 of SEQ ID NO: 24 may, independently, be substituted with Ala, Val, Leu, Ile, Pro, Phe, Met, or Trp; preferably with Leu, Ile, or Val. Preferably, all three of the native Ser111, Met113, and Gly114 of SEQ ID NO: 24 may, independently, be substituted with Ala, Val, Leu, Ile, Pro, Phe, Met, or Trp; preferably with Leu, Ile, or Val. In an aspect of the invention, the LVL-modified TCR comprises (i) SEQ ID NO: 21, (ii) SEQ ID NO: 22, (iii) SEQ ID NO: 23, (iv) both of SEQ ID NOs: 21 and 22, or (v) both of SEQ ID NOs: 21 and 23, wherein both of SEQ ID NOs: 21 and 22 or both of SEQ ID NOs: 21 and 23 are as defined in Table 2. The LVL-modified TCRs of the invention may include the substituted constant region in addition to any of the CDRs or variable regions described herein.

In an aspect of the invention, the LVL-modified TCR comprises a full length α chain and a full-length β chain. Examples of LVL-modified TCR α chain and β chain sequences are set forth in Table 2. In an aspect of the invention, the TCR comprises: (i) SEQ ID NO: 27, (ii) SEQ ID NO: 28, (iii) SEQ ID NO: 29, (iv) SEQ ID NO: 30, (v) SEQ ID NO: 35, (vi) SEQ ID NO: 36, (vii) SEQ ID NO: 37, (viii) SEQ ID NO: 38, (ix) both of SEQ ID NOs: 27 and 28, (x) both of SEQ ID NOs: 29 and 30, (xi) both of SEQ ID NOs: 35 and 36, (xii) both of SEQ ID NOs: 37 and 38, wherein all of SEQ ID NOs: 27-30 and SEQ ID NOs: 35-38 are as defined in Table 2.

TABLE 2

| SEQ ID | |
|---|---|
| NO: | Definitions of “X” |
| SEQ ID | X at position 47 is Thr; |
| NO: 21 | X at position 111 is Ser, Ala, Val, Leu, Ile, Pro, Phe, Met, |
| (constant | or Trp; preferably wherein X at position 111 is Leu, Ile, |
| region α | or Val; especially preferably wherein X at position 111 |
| chain) | is Leu; X at position 113 is Met, Ala, Val, Leu, Ile, Pro, Phe, |
| | or Trp; preferably wherein X at position 113 is Leu, Ile, |
| | or Val; especially preferably wherein X at position 113 |
| | is Ile; and X at position 114 is Gly, Ala, Val, Leu, Ile, |
| | Pro, Phe, Met, or Trp; preferably wherein X at position |
| | 114 is Leu, Ile, or Val; especially preferably wherein |
| | X at position 114 is Val; wherein SEQ ID NO: 21 does |
| | not comprise SEQ ID NO: 24 (unsubstituted α |
| | chain constant region) |
| SEQ ID | X at position 59 is Ser |
| NO: 22 | |
| (constant | |
| region β | |
| chain) | |
| SEQ ID | X at position 59 is Ser |
| NO: 23 | |
| (constant | |
| region β | |
| chain) | |
| SEQ ID | X at position 177 is Thr; |
| NO: 27 | X at position 241 is Ser, Ala, Val, Leu, Ile, Pro, Phe, Met, |
| (mTCR | or Trp; preferably wherein X at position 241 is Leu, Ile, |
| 4734_1 α | or Val; especially preferably wherein X at position 241 |
| chain) | is Leu; X at position 243 is Met, Ala, Val, Leu, Ile, Pro, Phe, |
| (with N- | or Trp; preferably wherein X at position 243 is Leu, Ile, |
| terminal | or Val; especially preferably wherein X at position 243 is |
| signal | Ile; and X at position 244 is Gly, Ala, Val, Leu, Ile, Pro, |
| peptide) | Phe, Met, or Trp;preferably wherein X at position 244 |
| | is Leu, Ile, or Val; especially preferably wherein X at |
| | position 244 is Val, wherein SEQ ID NO: 27 does not |
| | comprise SEQ ID NO: 24 (unsubstituted α chain |
| | constant region) |
| SEQ ID | X at position 191 is Ser |
| NO: 28 | |
| (mTCR | |
| 4734_1 β | |
| chain) | |
| (with N- | |
| terminal | |
| signal | |
| peptide) | |
| SEQ ID | X at position 157 is Thr; |
| NO: 29 | X at position 221 is Ser, Ala, Val, Leu, Ile, Pro, Phe, Met, |
| (mTCR | or Trp; preferably wherein X at position 221 is Leu, |
| 4734_1 α | Ile, or Val; especially preferably wherein X at position |
| chain) | 221 is Leu; X at position 223 is Met, Ala, Val, Leu, Ile, |
| (predicted | Pro, Phe, or Trp; preferably wherein X at position 223 |
| sequence | is Leu, Ile, or Val; especially preferably wherein X at |
| without N- | position 223 is Ile; and X at position 224 is Gly, Ala, |
| terminal | Val, Leu, Ile, Pro, Phe, Met, or Trp; preferably wherein |
| signal | X at position 224 is Leu, Ile, or Val; especially preferably |
| peptide) | wherein X at position 224 is Val, wherein SEQ ID |
| | NO: 29 does not comprise SEQ ID NO: 24 |
| | (unsubstituted α chain constant region) |
| SEQ ID | X at position 172 is Ser |
| NO: 30 | |
| (mTCR | |
| 4734_1 β | |
| chain) | |
| (predicted | |
| sequence | |
| without N- | |
| terminal | |
| signal | |
| peptide) | |
| SEQ ID | X at position 184 is Thr; |
| NO: 35 | X at position 248 is Ser, Ala, Val, Leu, Ile, Pro, Phe, Met, |
| (mTCR | or Trp; preferably wherein X at position 248 is Leu, Ile, |
| 5489_4 α | or Val; especially preferably wherein X at position 248 |
| chain) | is Leu; X at position 250 is Met, Ala, Val, Leu, Ile, Pro, Phe, |
| (with N- | or Trp; preferably wherein X at position 250 is Leu, Ile, |
| terminal | or Val; especially preferably wherein X at position 250 |
| signal | is Ile; and X at position 251 is Gly, Ala, Val, Leu, Ile, Pro, |
| peptide) | Phe, Met, or Trp; preferably wherein X at position 251 |
| | is Leu, Ile, or Val; especially preferably wherein X at |
| | position 251 is Val, wherein SEQ ID NO: 35 does not |
| | comprise SEQ ID NO: 24 (unsubstituted α |
| | chain constant region) |
| SEQ ID | X at position 188 is Ser |
| NO: 36 | |
| (mTCR | |
| 5489_4 β | |
| chain) | |
| (with N- | |
| terminal | |
| signal | |
| peptide) | |
| SEQ ID | X at position 163 is Thr; |
| NO: 37 | X at position 227 is Ser, Ala, Val, Leu, Ile, Pro, Phe, Met, |
| (mTCR | or Trp; preferably wherein X at position 227 is Leu, |
| 5489_4 α | Ile, or Val; especially preferably wherein X at position |
| chain) | 227 is Leu; |
| (predicted | X at position 229 is Met, Ala, Val, Leu, Ile, Pro, Phe, or Trp; |
| sequence | preferably wherein X at position 229 is Leu, Ile, or Val; |
| without N- | especially preferably wherein X at position 229 is Ile; and |
| terminal | X at position 230 is Gly, Ala, Val, Leu, Ile, Pro, Phe, Met, |
| signal | or Trp; preferably wherein X at position 230 is Leu, Ile, |
| peptide) | or Val; especially preferably wherein X at position 230 |
| | is Val, wherein SEQ ID NO: 37 does not comprise SEQ |
| | ID NO: 24 (unsubstituted α chain constant region) |
| SEQ ID | X at position 169 is Ser |
| NO: 38 | |
| (mTCR | |
| 5489_4 β | |
| chain) | |
| (predicted | |
| sequence | |
| without N- | |
| terminal | |
| signal | |
| peptide) | |

In an aspect of the invention, the substituted amino acid sequence includes the cysteine substitutions in the constant region of one or both of the α and β chains in combination with the substitution(s) of one, two, or three amino acids in the transmembrane (TM) domain of the constant region of the α chain with a hydrophobic amino acid (also referred to herein as “cysteine-substituted, LVL-modified TCR”). In this regard, the TCR is a cysteine-substituted, LVL-modified TCR in which the native Thr47 of SEQ ID NO: 24 is substituted with Cys; one, two, or three of the native Ser111, Met113, and Gly114 of SEQ ID NO: 24 are, independently, substituted with Ala, Val, Leu, Ile, Pro, Phe, Met, or Trp; preferably with Leu, Ile, or Val; and the native Ser59 of SEQ ID NO: 25 or SEQ ID NO: 26 is substituted with Cys. Preferably, all three of the native Ser111, Met113, and Gly114 of SEQ ID NO: 24 may, independently, be substituted with Ala, Val, Leu, Ile, Pro, Phe, Met, or Trp; preferably with Leu, Ile, or Val. In an aspect of the invention, the cysteine-substituted, LVL-modified TCR comprises (i) SEQ ID NO: 21, (ii) SEQ ID NO: 22, (iii) SEQ ID NO: 23, (iv) both of SEQ ID NOs: 21 and 22, or (v) both of SEQ ID NOs: 21 and 23, wherein both of SEQ ID NOs: 21 and 22 or both of SEQ ID NOs: 21 and 23 are as defined in Table 3. The cysteine-substituted, LVL-modified TCRs of the invention may include the substituted constant region in addition to any of the CDRs or variable regions described herein.

In an aspect, the cysteine-substituted, LVL-modified TCR comprises a full-length α chain and a full-length β chain. Examples of cysteine-substituted, LVL-modified TCR α chain and β chain sequences are set forth in Table 3. In an aspect of the invention, the TCR comprises: (i) SEQ ID NO: 27, (ii) SEQ ID NO: 28, (iii) SEQ ID NO: 29, (iv) SEQ ID NO: 30, (v) SEQ ID NO: 35, (vi) SEQ ID NO: 36, (vii) SEQ ID NO: 37, (viii) SEQ ID NO: 38, (ix) both of SEQ ID NOs: 27 and 28, (x) both of SEQ ID NOs: 29 and 30, (xi) both of SEQ ID NOs: 35 and 36, (xii) both of SEQ ID NOs: 37 and 38, wherein all of SEQ ID NOs: 27-30 and SEQ ID NOs: 35-38 are as defined in Table 3.

TABLE 3

| SEQ ID | |
|---|---|
| NO: | Definitions of “X” |
| SEQ ID | X at position 47 is Cys; |
| NO: 21 | X at position 111 is Ser, Ala, Val, Leu, Ile, Pro, Phe, Met, |
| (constant | or Trp; preferably wherein X at position 111 is Leu, |
| region α | Ile, or Val; especially preferably wherein X at position |
| chain) | 111 is Leu; X at position 113 is Met, Ala, Val, Leu, Ile, |
| | Pro, Phe, or Trp; preferably wherein X at position 113 |
| | is Leu, Ile, or Val; especially preferably wherein X at |
| | position 113 is Ile; and X at position 114 is Gly, Ala, |
| | Val, Leu, Ile, Pro, Phe, Met, or Trp; preferably wherein |
| | X at position 114 is Leu, Ile, or Val; and especially |
| | preferably wherein X at position 114 is Val, |
| | wherein SEQ ID NO: 21 does not simultaneously |
| | comprise all of Ser at position 111, Met at position |
| | 113, and Gly at position 114. |
| SEQ ID | X at position 59 is Cys |
| NO: 22 | |
| (constant | |
| region β | |
| chain) | |
| SEQ ID | X at position 59 is Cys |
| NO: 23 | |
| (constant | |
| region β | |
| chain) | |
| SEQ ID | X at position 177 is Cys; X at position 241 is Ser, Ala, |
| NO: 27 | Val, Leu, Ile, Pro, Phe, Met, or Trp; preferably wherein |
| (mTCR | X at position 241 is Leu, Ile, or Val; especially |
| 4734_1 α | preferably wherein X at position 241 is Leu; X at |
| chain) | position 243 is Met, Ala, Val, Leu, Ile, Pro, Phe, or Trp; |
| (with | preferably wherein X at position 243 is Leu, Ile, or Val; |
| N-terminal | especially preferably wherein X at position 243 is Ile; |
| signal | and X at position 244 is Gly, Ala, Val, Leu, Ile, Pro, Phe, |
| peptide) | Met, or Trp; preferably wherein X at position 244 is |
| | Leu, Ile, or Val; and especially preferably wherein X at |
| | position 244 is Val, wherein SEQ ID NO: 27 does not |
| | simultaneously comprise all of Ser at position 241, |
| | Met at position 243, and Gly at position 244. |
| SEQ ID | X at position 191 is Cys |
| NO: 28 | |
| (mTCR | |
| 4734_1 β | |
| chain) | |
| (with | |
| N-terminal | |
| signal | |
| peptide) | |
| SEQ ID | X at position 157 is Cys; X at position 221 is Ser, Ala, |
| NO: 29 | Val, Leu, Ile, Pro, Phe, Met, or Trp; preferably wherein |
| (mTCR | X at position 221 is Leu, Ile, or Val; especially preferably |
| 4734_1 α | wherein X at position 221 is Leu; X at position 223 |
| chain) | is Met, Ala, Val, Leu, Ile, Pro, Phe, or Trp; preferably |
| (predicted | wherein X at position 223 is Leu, Ile, or Val; especially |
| sequence | preferably wherein X at position 223 is Ile; and X at |
| without | position 224 is Gly, Ala, Val, Leu, Ile, Pro, Phe, Met, |
| N-terminal | or Trp; preferably wherein X at position 224 is Leu, |
| signal | Ile, or Val; and especially preferably wherein X at |
| peptide) | position 224 is Val, wherein SEQ ID NO: 29 does not |
| | simultaneously comprise all of Ser at position 221, |
| | Met at position 223, and Gly at position 224. |
| SEQ ID | X at position 172 is Cys |
| NO: 30 | |
| (mTCR | |
| 4734_1 β | |
| chain) | |
| (predicted | |
| sequence | |
| without | |
| N-terminal | |
| signal | |
| peptide) | |
| SEQ ID | X at position 184 is Cys; |
| NO: 35 | X at position 248 is Ser, Ala, Val, Leu, Ile, Pro, Phe, Met, |
| (mTCR | or Trp; preferably wherein X at position 248 is Leu, Ile, |
| 5489_4 α | or Val; especially preferably wherein X at position 248 |
| chain) | is Leu; X at position 250 is Met, Ala, Val, Leu, Ile, Pro, Phe, |
| (with | or Trp; preferably wherein X at position 250 is Leu, Ile, |
| N-terminal | or Val; especially preferably wherein X at position 250 |
| signal | is Ile; and X at position 251 is Gly, Ala, Val, Leu, Ile, Pro, |
| peptide) | Phe, Met, or Trp; preferably wherein X at position 251 |
| | is Leu, Ile, or Val; and especially preferably wherein |
| | X at position 251 is Val, wherein SEQ ID NO: 35 does |
| | not simultaneously comprise all of Ser at position 248, |
| | Met at position 250, and Gly at position 251. |
| SEQ ID | X at position 188 is Cys |
| NO: 36 | |
| (mTCR | |
| 5489_4 β | |
| chain) | |
| (with | |
| N-terminal | |
| signal | |
| peptide) | |
| SEQ ID | X at position 163 is Cys; |
| NO: 37 | X at position 227 is Ser, Ala, Val, Leu, Ile, Pro, Phe, Met, |
| (mTCR | or Trp; preferably wherein X at position 227 is Leu, Ile, |
| 5489_4 α | or Val; especially preferably wherein X at position 227 |
| chain) | is Leu; X at position 229 is Met, Ala, Val, Leu, Ile, Pro, |
| (predicted | Phe, or Trp; preferably wherein X at position 229 is Leu, |
| sequence | Ile, or Val; especially preferably wherein X at position |
| without | 229 is Ile; and X at position 230 is Gly, Ala, Val, Leu, Ile, |
| N-terminal | Pro, Phe, Met, or Trp; preferably wherein X at position |
| signal | 230 is Leu, Ile, or Val; and especially preferably wherein |
| peptide) | X at position 230 is Val, wherein SEQ ID NO: 37 does |
| | not simultaneously comprise all of Ser at position |
| | 227, Met at position 229, and Gly at position 230. |
| SEQ ID | X at position 169 is Cys |
| NO: 38 | |
| (mTCR | |
| 5489_4 β | |
| chain) | |
| (predicted | |
| sequence | |
| without | |
| N-terminal | |
| signal | |
| peptide) | |

Also provided by the invention is a polypeptide comprising a functional portion of any of the TCRs described herein. The term “polypeptide,” as used herein, includes oligopeptides and refers to a single chain of amino acids connected by one or more peptide bonds.

With respect to the inventive polypeptides, the functional portion can be any portion comprising contiguous amino acids of the TCR of which it is a part, provided that the functional portion specifically binds to V600E BRAF. The term “functional portion,” when used in reference to a TCR, refers to any part or fragment of the TCR, which part or fragment retains the biological activity of the TCR of which it is a part (the parent TCR). Functional portions encompass, for example, those parts of a TCR that retain the ability to specifically bind to V600E BRAF (e.g., presented by an HLA-A3 molecule), or detect, treat, or prevent cancer, to a similar extent, the same extent, or to a higher extent, as the parent TCR. In reference to the parent TCR, the functional portion can comprise, for instance, about 10%, about 25%, about 30%, about 50%, about 70%, about 80%, about 90%, about 95%, or more, of the parent TCR.

The functional portion can comprise additional amino acids at the amino or carboxy terminus of the portion, or at both termini, which additional amino acids are not found in the amino acid sequence of the parent TCR. Desirably, the additional amino acids do not interfere with the biological function of the functional portion, e.g., specifically binding to V600E BRAF; and/or having the ability to detect cancer, treat or prevent cancer, etc. More desirably, the additional amino acids enhance the biological activity, as compared to the biological activity of the parent TCR.

The polypeptide can comprise a functional portion of either or both of the α and β chains of the TCRs, such as a functional portion comprising one or more of the CDR1, CDR2, and CDR3 of the variable region(s) of the α chain and/or β chain of the TCR. In an aspect of the invention, the polypeptide can comprise the amino acid sequence of SEQ ID NO: 1 (CDR1 of α chain), SEQ ID NO: 2 (CDR2 of α chain), SEQ ID NO: 3 (CDR3 of α chain), SEQ ID NO: 4 (CDR1 of β chain), SEQ ID NO: 5 (CDR2 of β chain), SEQ ID NO: 6 (CDR3 of β chain), or a combination thereof. In an aspect of the invention, the polypeptide can comprise the amino acid sequence of SEQ ID NO: 11 (CDR1 of α chain), SEQ ID NO: 12 (CDR2 of α chain), SEQ ID NO: 13 (CDR3 of α chain), SEQ ID NO: 14 (CDR1 of β chain), SEQ ID NO: 15 (CDR2 of β chain), SEQ ID NO: 16 (CDR3 of β chain), or a combination thereof. In this regard, the inventive polypeptide can comprise any one or more of the amino acid sequences selected from the group consisting of SEQ ID NOs: 1-6 and SEQ ID NO: 11-16. In an aspect of the invention, the polypeptide comprises the amino acid sequences of: (a) all of SEQ ID NO: 1-3; (b) all of SEQ ID NO: 4-6; (c) all of SEQ ID NO: 1-6; (d) all of SEQ ID NO: 11-13; (e) all of SEQ ID NO: 14-16; or (f) all of SEQ ID NO: 11-16. In a preferred aspect, the polypeptide comprises the amino acid sequences of all of SEQ ID NOs: 1-6 or all of SEQ ID NOs: 11-16.

In an aspect of the invention, the polypeptide can comprise, for instance, the variable region of the inventive TCR comprising a combination of the CDR regions set forth above. In this regard, the polypeptide can comprise the amino acid sequence of (i) SEQ ID NO: 7, (ii) SEQ ID NO: 8, (iii) SEQ ID NO: 9, (iv) SEQ ID NO: 10, (v) both of SEQ ID NOs: 7 and 8, (vi) both of SEQ ID NOs: 9 and 10, (vii) SEQ ID NO: 17, (viii) SEQ ID NO: 18, (ix) SEQ ID NO: 19, (x) SEQ ID NO: 20, (xi) both of SEQ ID NOs: 17 and 18, or (xii) both of SEQ ID NOs: 19 and 20. Preferably, the polypeptide comprises the amino acid sequences of (i) both of SEQ ID NOs: 7 and 8, (ii) both of SEQ ID NOs: 9 and 10, (iii) both of SEQ ID NOs: 17 and 18, or (iv) both of SEQ ID NOs: 19 and 20.

In an aspect of the invention, the polypeptide can further comprise the constant region of the inventive TCR set forth above. In this regard, the polypeptide can further comprise the amino acid sequence of (i) SEQ ID NO: 24 (WT murine constant region of α chain), (ii) SEQ ID NO: 25 (WT murine constant region of β chain), (iii) SEQ ID NO: 26 (WT murine constant region of β chain), (iv) SEQ ID NO: 21 (substituted murine constant region of α chain), (v) SEQ ID NO: 22 (substituted murine constant region of β chain), (vi) SEQ ID NO: 23 (substituted murine constant region of β chain), (vii) both SEQ ID NOs: 21 and 22, (viii) both SEQ ID NOs: 21 and 23, (ix) both SEQ ID NO: 24 and 25, or (x) both SEQ ID NO: 24 and 26. Preferably, the polypeptide further comprises the amino acid sequences of (i) both SEQ ID NOs: 21 and 22, (ii) both SEQ ID NOs: 21 and 23, (iii) both SEQ ID NO: 24 and 25, or (iv) both SEQ ID NO: 24 and 26 in combination with any of the CDR regions or variable regions described herein with respect to other aspects of the invention. In an aspect of the invention, one or both of SEQ ID NOs: 21 and 22 of the polypeptide or one or both of SEQ ID NO: 21 and 23 of the polypeptide are as defined in any one of Tables 1-3.

In an aspect of the invention, the inventive polypeptide can comprise the entire length of an α or β chain of the TCR described herein. In this regard, the inventive polypeptide can comprise the amino acid sequence of SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, or SEQ ID NO: 42. Alternatively, the polypeptide of the invention can comprise both chains of the TCRs described herein. For example, the polypeptide may comprise both of SEQ ID NOs: 27 and 28, both of SEQ ID NOs: 29 and 30, both of SEQ ID NOs: 31 and 32, both of SEQ ID NOs: 33 and 34, both of SEQ ID NOs: 35 and 36, both of SEQ ID NOs: 37 and 38, both of SEQ ID NOs: 39 and 40, or both of SEQ ID NOs: 41 and 42.

For example, the polypeptide can comprise (a) an α chain comprising the amino acid sequence of SEQ ID NO: 27, wherein: (i) X at position 177 of SEQ ID NO: 27 is Thr or Cys; (ii) X at position 241 of SEQ ID NO: 27 is Ser, Ala, Val, Leu, Ile, Pro, Phe, Met, or Trp; (iii) X at position 243 of SEQ ID NO: 27 is Met, Ala, Val, Leu, Ile, Pro, Phe, or Trp; and (iv) X at position 244 of SEQ ID NO: 27 is Gly, Ala, Val, Leu, Ile, Pro, Phe, Met, or Trp; (b) a β chain comprising the amino acid sequence of SEQ ID NO: 28, wherein X at position 191 of SEQ ID NO: 28 is Ser or Cys; (c) both SEQ ID NOs: 27 and 28; (d) an α chain comprising the amino acid sequence of SEQ ID NO: 29, wherein: (i) X at position 157 of SEQ ID NO: 29 is Thr or Cys; (ii) X at position 221 of SEQ ID NO: 29 is Ser, Ala, Val, Leu, Ile, Pro, Phe, Met, or Trp; (iii) X at position 223 of SEQ ID NO: 29 is Met, Ala, Val, Leu, Ile, Pro, Phe, or Trp; and (iv) X at position 224 of SEQ ID NO: 29 is Gly, Ala, Val, Leu, Ile, Pro, Phe, Met, or Trp; (e) a β chain comprising the amino acid sequence of SEQ ID NO: 30, wherein X at position 172 of SEQ ID NO: 30 is Ser or Cys; (f) both SEQ ID NOs: 29 and 30; (g) SEQ ID NO: 31; (h) SEQ ID NO: 32; (i) SEQ ID NO: 33; (j) SEQ ID NO: 34; (k) both SEQ ID NOS: 31 and 32; (1) both SEQ ID NOs: 33 and 34; (m) an α chain comprising the amino acid sequence of SEQ ID NO: 35, wherein: (i) X at position 184 of SEQ ID NO: 35 is Thr or Cys; (ii) X at position 248 of SEQ ID NO: 35 is Ser, Ala, Val, Leu, Ile, Pro, Phe, Met, or Trp; (iii) X at position 250 of SEQ ID NO: 35 is Met, Ala, Val, Leu, Ile, Pro, Phe, or Trp; and (iv) X at position 251 of SEQ ID NO: 35 is Gly, Ala, Val, Leu, Ile, Pro, Phe, Met, or Trp; (n) a β chain comprising the amino acid sequence of SEQ ID NO: 36, wherein X at position 188 of SEQ ID NO: 36 is Ser or Cys; (o) both SEQ ID NOs: 35 and 36; (p) an α chain comprising the amino acid sequence of SEQ ID NO: 37, wherein: (i) X at position 163 of SEQ ID NO: 37 is Thr or Cys; (ii) X at position 227 of SEQ ID NO: 37 is Ser, Ala, Val, Leu, Ile, Pro, Phe, Met, or Trp; (iii) X at position 229 of SEQ ID NO: 37 is Met, Ala, Val, Leu, Ile, Pro, Phe, or Trp; and (iv) X at position 230 of SEQ ID NO: 37 is Gly, Ala, Val, Leu, Ile, Pro, Phe, Met, or Trp; (q) a β chain comprising the amino acid sequence of SEQ ID NO: 38, wherein X at position 169 of SEQ ID NO: 38 is Ser or Cys; (r) both SEQ ID NOs: 37 and 38; (s) SEQ ID NO: 39; (t) SEQ ID NO: 40; (u) SEQ ID NO: 41; (v) SEQ ID NO: 42; (w) both SEQ ID NOs: 39 and 40; or (x) both SEQ ID NOs: 41 and 42. In an aspect of the invention, any one or more of SEQ ID NOs: 27-30 and SEQ ID NOs: 35-38 of the polypeptide are as defined in any one of Tables 1-3.

An aspect of the invention further provides a protein comprising at least one of the polypeptides described herein. By “protein” is meant a molecule comprising one or more polypeptide chains.

In an aspect, the protein of the invention can comprise (a) a first polypeptide chain comprising the amino acid sequences of SEQ ID NOs: 1-3; (b) a second polypeptide chain comprising the amino acid sequences of SEQ ID NOs: 4-6; (c) a first polypeptide chain comprising the amino acid sequences of SEQ ID NOs: 1-3 and a second polypeptide chain comprising the amino acid sequences of SEQ ID NOs: 4-6; (d) a first polypeptide chain comprising the amino acid sequences of SEQ ID NOs: 11-13; (e) a second polypeptide chain comprising the amino acid sequences of SEQ ID NOs: 14-16; or (f) a first polypeptide chain comprising the amino acid sequences of SEQ ID NOs: 11-13 and a second polypeptide chain comprising the amino acid sequences of SEQ ID NOs: 14-16.

In another aspect of the invention, (a) the first polypeptide chain comprises the amino acid sequence of SEQ ID NO: 7; (b) the second polypeptide chain comprises the amino acid sequence of SEQ ID NO: 8; (c) the first polypeptide chain comprises the amino acid sequence of SEQ ID NO: 9; (d) the second polypeptide chain comprises the amino acid sequence of SEQ ID NO: 10; (e) the first polypeptide chain comprises the amino acid sequence of SEQ ID NO: 7 and the second polypeptide chain comprises the amino acid sequence of SEQ ID NO: 8; (f) the first polypeptide chain comprises the amino acid sequence of SEQ ID NO: 9 and the second polypeptide chain comprises the amino acid sequence of SEQ ID NO: 10; (g) the first polypeptide chain comprises the amino acid sequence of SEQ ID NO: 17; (h) the second polypeptide chain comprises the amino acid sequence of SEQ ID NO: 18; (i) the first polypeptide chain comprises the amino acid sequence of SEQ ID NO: 19; (j) the second polypeptide chain comprises the amino acid sequence of SEQ ID NO: 20; (k) the first polypeptide chain comprises the amino acid sequence of SEQ ID NO: 17 and the second polypeptide chain comprises the amino acid sequence of SEQ ID NO: 18; or (l) the first polypeptide chain comprises the amino acid sequence of SEQ ID NO: 19 and the second polypeptide chain comprises the amino acid sequence of SEQ ID NO: 20.

The inventive protein may further comprise any of the constant regions described herein with respect to other aspects of the invention. In this regard, in an aspect of the invention, (i) the first polypeptide chain may further comprise the amino acid sequence of SEQ ID NO: 21; (ii) the second polypeptide chain may further comprise the amino acid sequence of SEQ ID NO: 22; (iii) the second polypeptide chain may further comprise the amino acid sequence of SEQ ID NO: 23; (iv) the first polypeptide chain may further comprise the amino acid sequence of SEQ ID NO: 24; (v) the second polypeptide chain may further comprise the amino acid sequence of SEQ ID NO: 25; (vi) the second polypeptide chain may further comprise the amino acid sequence of SEQ ID NO: 26; (vii) the first polypeptide chain may further comprise the amino acid sequence of SEQ ID NO: 21 and the second polypeptide chain may further comprise the amino acid sequence of SEQ ID NO: 22; (viii) the first polypeptide chain may further comprise the amino acid sequence of SEQ ID NO: 21 and the second polypeptide chain may further comprise the amino acid sequence of SEQ ID NO: 23; (ix) the first polypeptide chain may further comprise the amino acid sequence of SEQ ID NO: 24 and the second polypeptide chain may further comprise the amino acid sequence of SEQ ID NO: 25; or (x) the first polypeptide chain may further comprise the amino acid sequence of SEQ ID NO: 24 and the second polypeptide chain may further comprise the amino acid sequence of SEQ ID NO: 26. In an aspect of the invention, one or both of SEQ ID NOs: 21 and 22 of the protein or one or both of SEQ ID NOs: 21 and 23 of the protein are as defined in any one of Tables 1-3.

The inventive protein may comprise a full length α or β chain, as described herein with respect to other aspects of the invention. In this regard, in an aspect of the invention, (a) the first polypeptide chain comprises the amino acid sequence of SEQ ID NO: 27, wherein: (i) X at position 177 of SEQ ID NO: 27 is Thr or Cys; (ii) X at position 241 of SEQ ID NO: 27 is Ser, Ala, Val, Leu, Ile, Pro, Phe, Met, or Trp; (iii) X at position 243 of SEQ ID NO: 27 is Met, Ala, Val, Leu, Ile, Pro, Phe, or Trp; and (iv) X at position 244 of SEQ ID NO: 27 is Gly, Ala, Val, Leu, Ile, Pro, Phe, Met, or Trp; (b) the second polypeptide chain comprises the amino acid sequence of SEQ ID NO: 28, wherein X at position 191 of SEQ ID NO: 28 is Ser or Cys; (c) the first polypeptide chain comprises the amino acid sequence of SEQ ID NOs: 27 and the second polypeptide chain comprises the amino acid sequence of SEQ ID NO: 28; (d) the first polypeptide chain comprises the amino acid sequence of SEQ ID NO: 29, wherein: (i) X at position 157 of SEQ ID NO: 29 is Thr or Cys; (ii) X at position 221 of SEQ ID NO: 29 is Ser, Ala, Val, Leu, Ile, Pro, Phe, Met, or Trp; (iii) X at position 223 of SEQ ID NO: 29 is Met, Ala, Val, Leu, Ile, Pro, Phe, or Trp; and (iv) X at position 224 of SEQ ID NO: 29 is Gly, Ala, Val, Leu, Ile, Pro, Phe, Met, or Trp; (e) the second polypeptide chain comprises the amino acid sequence of SEQ ID NO: 30, wherein X at position 172 of SEQ ID NO: 30 is Ser or Cys; (f) the first polypeptide chain comprises the amino acid sequence of SEQ ID NOs: 29 and the second polypeptide chain comprises the amino acid sequence of SEQ ID NO: 30; (g) the first polypeptide chain comprises the amino acid sequence of SEQ ID NO: 31; (h) the second polypeptide chain comprises the amino acid sequence of SEQ ID NO: 32; (i) the first polypeptide chain comprises the amino acid sequence of SEQ ID NO: 33; (j) the second polypeptide chain comprises the amino acid sequence of SEQ ID NO: 34; (k) the first polypeptide chain comprises the amino acid sequence of SEQ ID NOs: 31 and the second polypeptide chain comprises the amino acid sequence of SEQ ID NO: 32; (l) the first polypeptide chain comprises the amino acid sequence of SEQ ID NOs: 33 and the second polypeptide chain comprises the amino acid sequence of SEQ ID NO: 34; (m) the first polypeptide chain comprises the amino acid sequence of SEQ ID NO: 35, wherein: (i) X at position 184 of SEQ ID NO: 35 is Thr or Cys; (ii) X at position 248 of SEQ ID NO: 35 is Ser, Ala, Val, Leu, Ile, Pro, Phe, Met, or Trp; (iii) X at position 250 of SEQ ID NO: 35 is Met, Ala, Val, Leu, Ile, Pro, Phe, or Trp; and (iv) X at position 251 of SEQ ID NO: 35 is Gly, Ala, Val, Leu, Ile, Pro, Phe, Met, or Trp; (n) the second polypeptide chain comprises the amino acid sequence of SEQ ID NO: 36, wherein X at position 188 of SEQ ID NO: 36 is Ser or Cys; (o) the first polypeptide chain comprises the amino acid sequence of SEQ ID NOs: 35 and the second polypeptide chain comprises the amino acid sequence of SEQ ID NO: 36; (p) the first polypeptide chain comprises the amino acid sequence of SEQ ID NO: 37, wherein: (i) X at position 163 of SEQ ID NO: 37 is Thr or Cys; (ii) X at position 227 of SEQ ID NO: 37 is Ser, Ala, Val, Leu, Ile, Pro, Phe, Met, or Trp; (iii) X at position 229 of SEQ ID NO: 37 is Met, Ala, Val, Leu, Ile, Pro, Phe, or Trp; and (iv) X at position 230 of SEQ ID NO: 37 is Gly, Ala, Val, Leu, Ile, Pro, Phe, Met, or Trp; (q) the second polypeptide chain comprises the amino acid sequence of SEQ ID NO: 38, wherein X at position 169 of SEQ ID NO: 38 is Ser or Cys; (r) the first polypeptide chain comprises the amino acid sequence of SEQ ID NOs: 37 and the second polypeptide chain comprises the amino acid sequence of SEQ ID NO: 38; (s) the first polypeptide chain comprises the amino acid sequence of SEQ ID NO: 39; (t) the second polypeptide chain comprises the amino acid sequence of SEQ ID NO: 40; (u) the first polypeptide chain comprises the amino acid sequence of SEQ ID NO: 41; (v) the second polypeptide chain comprises the amino acid sequence of SEQ ID NO: 42; (w) the first polypeptide chain comprises the amino acid sequence of SEQ ID NOs: 39 and the second polypeptide chain comprises the amino acid sequence of SEQ ID NO: 40; or (x) the first polypeptide chain comprises the amino acid sequence of SEQ ID NOs: 41 and the second polypeptide chain comprises the amino acid sequence of SEQ ID NO: 42. In an aspect of the invention, one or more of SEQ ID NOs: 27-30 and SEQ ID NO: 35-38 are as defined in any one of Tables 1-3.

The protein of aspects of the invention can be a TCR. Alternatively, if, for example, the protein comprises a single polypeptide chain comprising the amino acid sequences of both the TCR α and β chains, or if the first and/or second polypeptide chain(s) of the protein further comprise(s) other amino acid sequences, e.g., an amino acid sequence encoding an immunoglobulin or a portion thereof, then the inventive protein can be a fusion protein. In this regard, the invention also provides a fusion protein comprising at least one of the inventive polypeptides described herein along with at least one other polypeptide. The other polypeptide can exist as a separate polypeptide of the fusion protein, or can exist as a polypeptide, which is expressed in frame (in tandem) with one of the inventive polypeptides described herein. The other polypeptide can encode any peptidic or proteinaceous molecule, or a portion thereof, including, but not limited to an immunoglobulin, CD3, CD4, CD8, an MHC molecule, a CD1 molecule, e.g., CD1a, CD1b, CD1c, CD1d, etc.

The fusion protein can comprise one or more copies of the inventive polypeptide and/or one or more copies of the other polypeptide. For instance, the fusion protein can comprise 1, 2, 3, 4, 5, or more, copies of the inventive polypeptide and/or of the other polypeptide. Suitable methods of making fusion proteins are known in the art, and include, for example, recombinant methods.

In some aspects of the invention, the TCRs, polypeptides, and proteins of aspects of the invention may be expressed as a single protein comprising a linker peptide linking the α chain and the β chain. In this regard, the TCRs, polypeptides, and proteins may further comprise a linker peptide. The linker peptide may advantageously facilitate the expression of a recombinant TCR, polypeptide, and/or protein in a host cell. The linker peptide may comprise any suitable amino acid sequence. The linker peptide may be a cleavable linker peptide. For example, the linker peptide may be a furin-SGSG-P2A linker peptide comprising the amino acid sequence of RAKRSGSGATNFSLLKQAGDVEENPGP (SEQ ID NO: 43). Upon expression of the construct including the linker peptide by a host cell, the linker peptide may be cleaved, resulting in separated α and β chains. In an aspect of the invention, the TCR, polypeptide, or protein may comprise an amino acid sequence comprising a full-length α chain, a full-length β chain, and a linker peptide positioned between the α and β chains.

The protein of aspects of the invention can be a recombinant antibody, or an antigen binding portion thereof, comprising at least one of the inventive polypeptides described herein. As used herein, “recombinant antibody” refers to a recombinant (e.g., genetically engineered) protein comprising at least one of the inventive polypeptides and a polypeptide chain of an antibody, or an antigen binding portion thereof. The polypeptide of an antibody, or antigen binding portion thereof, can be a heavy chain, a light chain, a variable or constant region of a heavy or light chain, a single chain variable fragment (scFv), or an Fc, Fab, or F(ab)₂′ fragment of an antibody, etc. The polypeptide chain of an antibody, or an antigen binding portion thereof, can exist as a separate polypeptide of the recombinant antibody. Alternatively, the polypeptide chain of an antibody, or an antigen binding portion thereof, can exist as a polypeptide, which is expressed in frame (in tandem) with the inventive polypeptide. The polypeptide of an antibody, or an antigen binding portion thereof, can be a polypeptide of any antibody or any antibody fragment, including any of the antibodies and antibody fragments described herein.

Included in the scope of the invention are functional variants of the inventive TCRs, polypeptides, or proteins described herein. The term “functional variant,” as used herein, refers to a TCR, polypeptide, or protein having substantial or significant sequence identity or similarity to a parent TCR, polypeptide, or protein, which functional variant retains the biological activity of the TCR, polypeptide, or protein of which it is a variant. Functional variants encompass, for example, those variants of the TCR, polypeptide, or protein described herein (the parent TCR, polypeptide, or protein) that retain the ability to specifically bind to the V600E BRAF for which the parent TCR has antigenic specificity or to which the parent polypeptide or protein specifically binds, to a similar extent, the same extent, or to a higher extent, as the parent TCR, polypeptide, or protein. In reference to the parent TCR, polypeptide, or protein, the functional variant can, for instance, be at least about 30%, about 50%, about 75%, about 80%, about 90%, about 95%, about 96%, about 97%, about 98%, about 99% or more identical in amino acid sequence to the parent TCR, polypeptide, or protein, respectively.

The functional variant can, for example, comprise the amino acid sequence of the parent TCR, polypeptide, or protein with at least one conservative amino acid substitution. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

Alternatively or additionally, the functional variants can comprise the amino acid sequence of the parent TCR, polypeptide, or protein with at least one non-conservative amino acid substitution. In this case, it is preferable for the non-conservative amino acid substitution to not interfere with or inhibit the biological activity of the functional variant. Preferably, the non-conservative amino acid substitution enhances the biological activity of the functional variant, such that the biological activity of the functional variant is increased as compared to the parent TCR, polypeptide, or protein.

The TCR, polypeptide, or protein can consist essentially of the specified amino acid sequence or sequences described herein, such that other components of the TCR, polypeptide, or protein, e.g., other amino acids, do not materially change the biological activity of the TCR, polypeptide, or protein. In this regard, the inventive TCR, polypeptide, or protein can, for example, consist essentially of the amino acid sequence of SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, both of SEQ ID NOs: 27 and 28, both of SEQ ID NOs: 29 and 30, both of SEQ ID NOs: 31 and 32, both of SEQ ID NOs: 33 and 34, both of SEQ ID NOs: 35 and 36, both of SEQ ID NOs: 37 and 38, both of SEQ ID NOs: 39 and 40, or both of SEQ ID NOs: 41 and 42. Also, for instance, the inventive TCRs, polypeptides, or proteins can consist essentially of the amino acid sequence(s) of (i) SEQ ID NO: 7, (ii) SEQ ID NO: 8, (iii) SEQ ID NO: 9, (iv) SEQ ID NO: 10, (v) both of SEQ ID NOs: 7 and 8, (vi) both of SEQ ID NOs: 9 and 10, (vii) SEQ ID NO: 17, (viii) SEQ ID NO: 18, (ix) SEQ ID NO: 19, (x) SEQ ID NO: 20, (xi) both of SEQ ID NOs: 17 and 18, or (xii) both of SEQ ID NOs: 19 and 20. Furthermore, the inventive TCRs, polypeptides, or proteins can consist essentially of the amino acid sequences of (a) all of SEQ ID NO: 1-3; (b) all of SEQ ID NO: 4-6; (c) all of SEQ ID NO: 1-6; (d) all of SEQ ID NO: 11-13; (e) all of SEQ ID NO: 14-16; or (f) all of SEQ ID NO: 11-16.

The TCRs, polypeptides, and proteins of aspects of the invention can be of any length, i.e., can comprise any number of amino acids, provided that the TCRs, polypeptides, or proteins retain their biological activity, e.g., the ability to specifically bind to V600E BRAF; detect cancer in a mammal; or treat or prevent cancer in a mammal, etc. For example, the polypeptide can be in the range of from about 50 to about 5000 amino acids long, such as about 50, about 70, about 75, about 100, about 125, about 150, about 175, about 200, about 300, about 400, about 500, about 600, about 700, about 800, about 900, about 1000 or more amino acids in length. In this regard, the polypeptides of the invention also include oligopeptides.

The TCRs, polypeptides, and proteins of aspects of the invention can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, cx-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α, γ-diaminobutyric acid, α,β-diaminopropionic acid, homophenylalanine, and α-tert-butylglycine.

The TCRs, polypeptides, and proteins of aspects of the invention can be glycosylated, amidated, carboxylated, phosphorylated, esterified, N-acylated, cyclized via, e.g., a disulfide bridge, or converted into an acid addition salt and/or optionally dimerized or polymerized, or conjugated.

The inventive TCRs, polypeptides, and proteins described herein (including any of the functional portions or variants thereof) are also contemplated to be useful as the soluble TCR component of bispecific engager TCR fusion proteins (e.g., IMMTAC (immune-mobilizing monoclonal TCRs against cancer) molecules). Bispecific engager TCR fusion proteins have two components. One component comprises a soluble TCR. The other component comprises an anti-CD3 effector. The anti-CD3 effector may be any molecule that engages with a CD3 molecule on T cells and activates a T cell immune response. For example, the anti-CD3 effector may be an anti-CD3 antibody or anti-CD3 antibody fragment. The soluble TCR component of the bispecific engager TCR fusion protein binds to the target antigen presented on the surface of cancer cells presented by an HLA molecule. The anti-CD3 effector component engages a CD3 molecule on T cells. The engagement of these components of the bispecific engager TCR fusion protein triggers the activation and recruitment of T cells and redirects T-cell killing to tumor cells. An aspect of the invention provides a bispecific engager TCR fusion protein comprising (i) any of the inventive TCRs, polypeptides, or proteins (including any of the functional portions or variants thereof) described herein and (ii) an anti-CD3 engager. Hereinafter, references to “protein(s)” also encompass the bispecific engager TCR fusion proteins described herein, unless specified otherwise.

The TCR, polypeptide, and/or protein of aspects of the invention can be obtained by methods known in the art such as, for example, de novo synthesis. Also, polypeptides and proteins can be recombinantly produced using the nucleic acids described herein using standard recombinant methods. See, for instance, Green and Sambrook, Molecular Cloning: A Laboratory Manual, 4^{th} ed., Cold Spring Harbor Press, Cold Spring Harbor, NY (2012). Alternatively, the TCRs, polypeptides, and/or proteins described herein can be synthesized by any of a variety of commercial entities. In this respect, the inventive TCRs, polypeptides, and proteins can be synthetic, recombinant, isolated, and/or purified.

Included in the scope of the invention are conjugates, e.g., bioconjugates, comprising any of the inventive TCRs, polypeptides, or proteins (including any of the functional portions or variants thereof), nucleic acids, recombinant expression vectors, host cells, or populations of host cells. Conjugates, as well as methods of synthesizing conjugates in general, are known in the art.

An aspect of the invention provides a nucleic acid comprising a nucleotide sequence encoding any of the TCRs, polypeptides, proteins, or bispecific engager TCR fusion proteins described herein. “Nucleic acid,” as used herein, includes “polynucleotide,” “oligonucleotide,” and “nucleic acid molecule,” and generally means a polymer of DNA or RNA, which can be single-stranded or double-stranded, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In an aspect, the nucleic acid comprises complementary DNA (cDNA). It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

Preferably, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. See, for example, Green and Sambrook et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxy methylaminomethyl-2-thiouridine, 5-carboxymethylaminomethyluracil, dihydrouracil, β-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, β-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be synthesized by any of a variety of commercial entities.

The nucleic acid can comprise any nucleotide sequence which encodes any of the TCRs, polypeptides, or proteins described herein. In an aspect of the invention, the nucleic acid comprises a codon-optimized nucleotide sequence encoding any of the TCRs, polypeptides, or proteins described herein. Without being bound to any particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency.

An aspect of the invention provides an isolated or purified nucleic acid comprising, from 5′ to 3′, a first nucleic acid sequence and a second nucleotide sequence, wherein the first and second nucleotide sequence, respectively, encode the amino sequences of SEQ ID NOs: 7 and 8; 8 and 7; 9 and 10; 10 and 9; 17 and 18; 18 and 17; 19 and 20; 20 and 19; 27 and 28; 28 and 27; 29 and 30; 30 and 29; 31 and 32; 32 and 31; 33 and 34; 34 and 33; 35 and 36; 36 and 35; 37 and 38; 38 and 37; 39 and 40; 40 and 39; 41 and 42; or 42 and 41.

In an aspect of the invention, the isolated or purified nucleic acid further comprises a third nucleotide sequence interposed between the first and second nucleotide sequence, wherein the third nucleotide sequence encodes a cleavable linker peptide. In an aspect of the invention, the cleavable linker peptide comprises the amino acid sequence of RAKRSGSGATNFSLLKQAGDVEENPGP (SEQ ID NO: 43).

The nucleic acids of the invention can be incorporated into a recombinant expression vector. In this regard, the invention provides a recombinant expression vector comprising any of the nucleic acids of the invention. In an aspect of the invention, the recombinant expression vector comprises a nucleotide sequence encoding the α chain, the β chain, and linker peptide.

For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotide, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages do not hinder the transcription or replication of the vector.

The recombinant expression vector of the invention can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA). Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-Cl, pMAM and pMAMneo (Clontech). Preferably, the recombinant expression vector is a viral vector, e.g., a retroviral vector. In an especially preferred aspect, the recombinant expression vector is an MSGV1 vector. In an aspect of the invention, the recombinant expression vector is a transposon or a lentiviral vector.

The recombinant expression vectors of the invention can be prepared using standard recombinant DNA techniques described in, for example, Green and Sambrook et al., supra. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papillomavirus, and the like.

Desirably, the recombinant expression vector comprises regulatory sequences, such as transcription and translation initiation and termination codons, which are specific to the type of host cell (e.g., bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected host cells. Marker genes include biocide resistance, e.g., resistance to antibiotics, heavy metals, etc., complementation in an auxotrophic host cell to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the TCR, polypeptide, or protein. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, nitroreductase, and the inducible caspase 9 gene system.

Another aspect of the invention further provides a host cell comprising any of the nucleic acids or recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell is preferably a prokaryotic cell, e.g., a DH5α cell. For purposes of producing a recombinant TCR, polypeptide, or protein, the host cell is preferably a mammalian cell. Most preferably, the host cell is a human cell. While the host cell can be of any cell type, can originate from any type of tissue, and can be of any developmental stage, the host cell preferably is a peripheral blood lymphocyte (PBL) or a peripheral blood mononuclear cell (PBMC). More preferably, the host cell is a T cell. In an aspect of the invention, the host cell is a human lymphocyte. In another aspect of the invention, the host cell is selected from the group consisting of a T cell, a natural killer T (NKT) cell, an invariant natural killer T (iNKT) cell, a natural killer (NK) cell, a macrophage, a pluripotent cell, and a multipotent cell.

For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. Preferably, the T cell is a human T cell. The T cell can be any type of T cell and can be of any developmental stage, including but not limited to, CD4⁺/CD8⁺ double positive T cells, CD4⁺ helper T cells, e.g., Th₁ and Th₂ cells, CD4⁺ T cells, CD8⁺ T cells (e.g., cytotoxic T cells), tumor infiltrating lymphocytes (TILs), memory T cells (e.g., central memory T cells and effector memory T cells), naïve T cells, and the like.

In an aspect of the invention, the host cell is a pluripotent cell or a multipotent cell. Pluripotent cells have the capacity to give rise to any of the three germ layers: endoderm, mesoderm, and ectoderm. Pluripotent cells may comprise, for example, stem cells, e.g., embryonic stem cells, nuclear transfer derived embryonic stem cells, induced pluripotent stem cells (iPSC), etc. Multipotent cells may comprise, for example, hematopoietic stem cells. Modifying, e.g., reprogramming, cells to a pluripotent state refers to the reversion of a cell to a pluripotent cell and is described for example, in Crompton et al., Trends Immunol., 35 (4): 178-185 (2014). Exemplary techniques may include somatic cell nuclear transfer (SCNT), cell-cell fusion, and direct reprogramming. Examples of methods for carrying out cell-cell fusion are described, for example, in Ogle et al., Nat. Rev. Mol. Cell Biol. 6:567-75 (2005) and Zhou et al., Cell Stem Cell, 3:382-388 (2008). Examples of methods for carrying out SCNT are described, for example, in Hanna et al., Cell, 143:508-525 (2010); Stadtfeld et al., Genes Dev., 24:2239-2263 (2010); Wilmut et al., Nature, 385:810-813 (1997); Vizcardo et al., Cell Stem Cell, 12:31-36 (2013); and Crompton et al., Cell Stem Cell, 12:6-8 (2013). In an aspect of the invention, the host cell is an iPSC that was prepared by reprogramming, any of the host cells described herein (e.g., T cells, NK cells, or invariant natural killer T cells) to a pluripotent state.

Also provided by the invention is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a a neutrophil, an erythrocyte, a hepatocyte, an endothelial cell, an epithelial cells, a muscle cell, a brain cell, etc. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one aspect of the invention, the population of cells is a clonal population comprising host cells comprising a recombinant expression vector as described herein.

In an aspect of the invention, the numbers of cells in the population may be rapidly expanded. Expansion of the numbers of T cells can be accomplished by any of a number of methods as are known in the art as described in, for example, U.S. Pat. Nos. 8,034,334; 8,383,099; 11,401,503; Dudley et al., J. Immunother., 26:332-42 (2003); and Riddell et al., J. Immunol. Methods, 128:189-201 (1990). In an aspect, expansion of the numbers of T cells is carried out by culturing the T cells with OKT3 antibody, IL-2, and feeder PBMC (e.g., irradiated allogeneic PBMC).

The inventive TCRs, polypeptides, proteins, nucleic acids, recombinant expression vectors, and host cells (including populations thereof), can be isolated and/or purified. The term “isolated,” as used herein, means having been removed from its natural environment. The term “purified,” as used herein, means having been increased in purity, wherein “purity” is a relative term, and not to be necessarily construed as absolute purity. For example, the purity can be at least about 50%, can be greater than about 60%, about 70%, about 80%, about 90%, about 95%, or can be about 100%.

Still another aspect of the invention provides a method of producing a host cell expressing a TCR that has antigenic specificity for the peptide of KIGDFGLATEK (SEQ ID NO: 45), the method comprising contacting a cell with any of the vectors described herein under conditions that allow introduction of the vector into the cell.

Still another aspect of the invention provides a method of producing any of the TCRs, polypeptides, or proteins described herein, the method comprising culturing any of the host cells or populations of host cells described herein so that the TCR, polypeptide, or protein is produced.

An aspect of the invention provides an isolated or purified TCR, polypeptide, or protein encoded by any of the nucleic acids or vectors described herein with respect to other aspects of the invention.

Another aspect of the invention provides an isolated or purified TCR, polypeptide, or protein that results from expression of any of the nucleic acids or vectors described herein with respect to other aspects of the invention in a cell.

The inventive TCRs, polypeptides, proteins, bispecific engager TCR fusion proteins, nucleic acids, recombinant expression vectors, and host cells (including populations thereof), all of which are collectively referred to as “inventive TCR materials” hereinafter, can be formulated into a composition, such as a pharmaceutical composition. In this regard, the invention provides a pharmaceutical composition comprising any of the TCRs, polypeptides, proteins, bispecific engager TCR fusion proteins, nucleic acids, expression vectors, or host cells (including populations thereof), described herein, and a pharmaceutically acceptable carrier. The inventive pharmaceutical compositions containing any of the inventive TCR materials can comprise more than one inventive TCR material, e.g., a polypeptide and a nucleic acid, or two or more different TCRs. Alternatively, the pharmaceutical composition can comprise an inventive TCR material in combination with another pharmaceutically active agent(s) or drug(s), such as a chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

Preferably, the carrier is a pharmaceutically acceptable carrier. With respect to pharmaceutical compositions, the carrier can be any of those conventionally used for the particular inventive TCR material under consideration. Methods for preparing administrable compositions are known or apparent to those skilled in the art and are described in more detail in, for example, Remington: The Science and Practice of Pharmacy, 23rd Ed., Academic Press (2020). It is preferred that the pharmaceutically acceptable carrier be one which has no detrimental side effects or toxicity under the conditions of use.

The choice of carrier will be determined in part by the particular inventive TCR material, as well as by the particular method used to administer the inventive TCR material. Accordingly, there are a variety of suitable formulations of the pharmaceutical composition of the invention. Suitable formulations may include any of those for parenteral, subcutaneous, intravenous, intramuscular, intraarterial, intrathecal, intratumoral, or interperitoneal administration. More than one route can be used to administer the inventive TCR materials, and in certain instances, a particular route can provide a more immediate and more effective response than another route.

Preferably, the inventive TCR material is administered by injection, e.g., intravenously. When the inventive TCR material is a host cell (or population thereof) expressing the inventive TCR, the pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter, Deerfield, IL), about 5% dextrose in water, or Ringer's lactate. In an aspect, the pharmaceutically acceptable carrier is supplemented with human serum albumen.

For purposes of the invention, the amount or dose (e.g., numbers of cells when the inventive TCR material is one or more cells) of the inventive TCR material administered should be sufficient to effect, e.g., a therapeutic or prophylactic response, in the subject or animal over a reasonable time frame. For example, the dose of the inventive TCR material should be sufficient to bind to a cancer antigen (e.g., V600E BRAF), or detect, treat or prevent cancer in a period of from about 2 hours or longer, e.g., 12 to 24 or more hours, from the time of administration. In certain aspects, the time period could be even longer. The dose will be determined by the efficacy of the particular inventive TCR material and the condition of the animal (e.g., human), as well as the body weight of the animal (e.g., human) to be treated.

Many assays for determining an administered dose are known in the art. For purposes of the invention, an assay, which comprises comparing the extent to which target cells are lysed or IFN-γ is secreted by T cells expressing the inventive TCR, polypeptide, or protein upon administration of a given dose of such T cells to a mammal among a set of mammals of which each is given a different dose of the T cells, could be used to determine a starting dose to be administered to a mammal. The extent to which target cells are lysed or IFN-γ is secreted upon administration of a certain dose can be assayed by methods known in the art.

The dose of the inventive TCR material also will be determined by the existence, nature and extent of any adverse side effects that might accompany the administration of a particular inventive TCR material. Typically, the attending physician will decide the dosage of the inventive TCR material with which to treat each individual patient, taking into consideration a variety of factors, such as age, body weight, general health, diet, sex, inventive TCR material to be administered, route of administration, and the severity of the cancer being treated. In an aspect in which the inventive TCR material is a population of cells, the number of cells administered per infusion may vary, e.g., from about 1×10⁶ to about 1×10¹² cells or more. In certain aspects, fewer than 1×10⁶ cells may be administered.

One of ordinary skill in the art will readily appreciate that the inventive TCR materials of the invention can be modified in any number of ways, such that the therapeutic or prophylactic efficacy of the inventive TCR materials is increased through the modification. For instance, the inventive TCR materials can be conjugated either directly or indirectly through a bridge to a chemotherapeutic agent. The practice of conjugating compounds to a chemotherapeutic agent is known in the art. One of ordinary skill in the art recognizes that sites on the inventive TCR materials, which are not necessary for the function of the inventive TCR materials, are suitable sites for attaching a bridge and/or a chemotherapeutic agent, provided that the bridge and/or chemotherapeutic agent, once attached to the inventive TCR materials, do(es) not interfere with the function of the inventive TCR materials, i.e., the ability to bind to V600E BRAF or to detect, treat, or prevent cancer.

It is contemplated that the inventive pharmaceutical compositions, TCRs, polypeptides, proteins, bispecific engager TCR fusion proteins, nucleic acids, recombinant expression vectors, host cells, and populations of cells can be used in methods of treating or preventing cancer. Without being bound to a particular theory, the inventive TCRs are believed to bind specifically to V600E BRAF, such that the TCR (or related inventive polypeptide or protein), when expressed by a cell, is able to mediate an immune response against a target cell expressing V600E BRAF. In this regard, an aspect of the invention provides a method of treating or preventing cancer in a mammal, comprising administering to the mammal any of the pharmaceutical compositions, TCRs, polypeptides, proteins, or bispecific engager TCR fusion proteins described herein, any nucleic acid or recombinant expression vector comprising a nucleotide sequence encoding any of the TCRs, polypeptides, proteins, or bispecific engager TCR fusion proteins described herein, or any host cell or population of cells comprising a recombinant vector which encodes any of the TCRs, polypeptides, proteins, bispecific engager TCR fusion proteins described herein, in an amount effective to treat or prevent cancer in the mammal.

An aspect of the invention provides a method of inducing an immune response against a cancer in a mammal, comprising administering to the mammal any of the pharmaceutical compositions, TCRs, polypeptides, proteins, or bispecific engager TCR fusion proteins described herein, any nucleic acid or recombinant expression vector comprising a nucleotide sequence encoding any of the TCRs, polypeptides, proteins, or bispecific engager TCR fusion proteins described herein, or any host cell or population of cells comprising a recombinant vector which encodes any of the TCRs, polypeptides, proteins, or bispecific engager TCR fusion proteins described herein, in an amount effective to induce an immune response against the cancer in the mammal.

An aspect of the invention provides any of the pharmaceutical compositions, TCRs, polypeptides, proteins, or bispecific engager TCR fusion proteins described herein, any nucleic acid or recombinant expression vector comprising a nucleotide sequence encoding any of the TCRs, polypeptides, proteins, or bispecific engager TCR fusion proteins described herein, or any host cell or population of cells comprising a recombinant vector which encodes any of the TCRs, polypeptides, or proteins or bispecific engager TCR fusion proteins described herein, for use in the treatment or prevention of cancer in a mammal.

An aspect of the invention provides any of the pharmaceutical compositions, TCRs, polypeptides, proteins, or bispecific engager TCR fusion proteins, described herein, any nucleic acid or recombinant expression vector comprising a nucleotide sequence encoding any of the TCRs, polypeptides, proteins, or bispecific engager TCR fusion proteins described herein, or any host cell or population of cells comprising a recombinant vector which encodes any of the TCRs, polypeptides, proteins, or bispecific engager TCR fusion proteins described herein, for use in inducing an immune response against a cancer in a mammal.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the cancer being treated or prevented. For example, treatment or prevention can include promoting the regression of a tumor. Also, for purposes herein, “prevention” can encompass delaying the onset of the cancer, or a symptom or condition thereof. Alternatively or additionally, “prevention” may encompass preventing or delaying the recurrence of cancer, or a symptom or condition thereof.

Also provided is a method of detecting the presence of cancer in a mammal. The method comprises (i) contacting a sample comprising one or more cells from the mammal with any of the inventive TCRs, polypeptides, proteins, bispecific engager TCR fusion proteins, nucleic acids, recombinant expression vectors, host cells, populations of cells, or pharmaceutical compositions described herein, thereby forming a complex, and (ii) detecting the complex, wherein detection of the complex is indicative of the presence of cancer in the mammal.

With respect to the inventive method of detecting cancer in a mammal, the sample of cells can be a sample comprising whole cells, lysates thereof, or a fraction of the whole cell lysates, e.g., a nuclear or cytoplasmic fraction, a whole protein fraction, or a nucleic acid fraction.

For purposes of the inventive method of detecting cancer, the contacting can take place in vitro or in vivo with respect to the mammal. Preferably, the contacting is in vitro.

Also, detection of the complex can occur through any number of ways known in the art. For instance, the inventive TCRs, polypeptides, proteins, nucleic acids, recombinant expression vectors, host cells, or populations of cells, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

For purposes of the inventive methods, wherein host cells or populations of cells are administered, the cells can be cells that are allogeneic or autologous to the mammal. Preferably, the cells are autologous to the mammal.

With respect to the inventive methods, the cancer can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, chronic lymphocytic leukemia, chronic myeloid cancer, colon cancer, colorectal cancer, endometrial cancer, esophageal cancer, uterine cervical cancer, gastrointestinal carcinoid tumor, glioma, hairy cell leukemia, Hodgkin lymphoma, hypopharynx cancer, kidney cancer, larynx cancer, liver cancer, lung cancer (e.g., lung adenocarcinoma), malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, and urinary bladder cancer. A preferred cancer is melanoma, hairy cell leukemia, non-Hodgkin lymphoma, thyroid cancer, ovarian cancer, lung adenocarcinoma, colorectal cancer, or brain cancer. In an aspect of the invention, the cancer expresses a human BRAF^{V600E} amino acid sequence.

The mammal referred to in the inventive methods can be any mammal. As used herein, the term “mammal” refers to any mammal, including, but not limited to, mammals of the order Rodentia, such as mice and hamsters, and mammals of the order Lagomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perissodactyla, including Equines (horses). It is most preferred that the mammals are of the order Primates or Ceboids (monkeys) or of the order Anthropoids (humans and apes). An especially preferred mammal is the human.

The following examples further illustrate the invention but, of course, should not be construed as in any way limiting its scope.

### Example 1

This example demonstrates the isolation of two TCRs having antigenic specificity for V600E BRAF presented by HLA-A3.

### Immunization of HLA-A3/Kb Transgenic Mice

A 11-mer V600E BRAF peptide (KIGDFGLATEK) (SEQ ID NO: 45) was detected on HLA-A*03:01 complexes using mass spectrometric analysis. The 11-mer V600E BRAF peptide and the helper peptide TPPAYRPPNAPIL (SEQ ID NO: 44) (derived from HBV core protein amino acid residues 128-140) (120 μg) emulsified in Complete Freund's adjuvant were injected into the footpads of HLA-A*03:01/Kb transgenic mice. This initial immunization was followed by two biweekly subcutaneous administrations of the same dose of peptides emulsified in Complete Freund's adjuvant in the tail base.

### In Vitro Stimulation of T Cells

One week after the last immunization, splenocytes were harvested from the mice and stimulated in vitro with 1 μg/ml of (KIGDFGLATEK) (SEQ ID NO: 45) peptide in medium containing recombinant human IL-2. A second round of the same stimulation was performed if the antigen-specific T cells were not obtained after the first round.

### Screening of Antigen-Specific T Cells

T cells from in vitro-stimulated populations were co-cultured with either peptide pulsed targets or gene-engineered targets. IFN-gamma release from T cells were measured by ELISA (enzyme-linked immunosorbent assay). Positive populations were then subjected to KIGDFGLATEK (SEQ ID NO: 45) peptide/HLA-A3 tetramer binding and fluorescence-activated cell sorting (FACS).

### Molecular Cloning of Antigen-Specific TCRs

Total RNA was isolated from tetramer positive single cells and 5′ RACE-ready cDNAs were made. PCR products from primers derived from 5′ RACE (rapid amplification of cDNA ends) and mouse TCR constant chains were sequenced. TCR pairs were reconstituted based on the sequencing data.

The WT amino acid sequences of the isolated, full-length TCR alpha and beta chains are set forth in Table 4. In Table 4, the N-terminal signal peptides are in bold font, the CDRs are underlined, and the constant regions are italicized.

TABLE 4

| | Amino acid sequences |
|---|---|
| TCR chain | (N-terminus-CDR1, CDR2, CDR3-C terminus) |
| mTCR 4734_1 alpha chain | MQRNLGAVLGILWVQICWVSGDKVKQSPSALSLQE |
| (mouse TRAV4-3*03/ | GTNSALRCNFSIAATTVQWFLQNPRGSLINLFYLVPGT |
| TRAJ48*01) | KENGRLKSAFDSKESYSTLHIRDAQLEDSGTYFCAAH |
| (with N-terminal signal | GYGNEKITFGAGTKLTIKPNIQNPEPAVYQLKDPRSQDS |
| peptide) | TLCLFTDFDSQINVPKTMESGTFITDKTVLDMKAMDSKS |
| | NGAIAWSNQTSFTCQDIFKETNATYPSSDVPCDATLTEKS |
| | FETDMNLNFQNLSVMGLRILLLKVAGFNLLMTLRLWSS |
| | (SEQ ID NO: 31) |
| mTCR 4734_1 beta chain | MDIWLLGWIIFSFLEAGHTGPKVLQIPSHQIIDMGQM |
| (mouse TRBV3*01/ | VTLNCDPVSNHLYFYWYKQILGQQMEFLVNFYNGKV |
| TRBJ1-4*02/TRBD1*01) | MEKSKLFKDQFSVERPDGSYFTLKIQPTALEDSAVYFC |
| (with N-terminal signal | ASSSRDRGRERLFFGHGTKLSVLEDLRNVTPPKVSLFEP |
| peptide) | SKAELANKQKATLVCLARGFFPDHVELSWWVNGKEVHS |
| | GVSTDPQAYKESNYSYCLSSRLRVSATFWHNPRNHFRCQ |
| | VQFHGLSEEDKWPEGSPKPVTQNISAEAWGRADCGITSA |
| | SYQQGVLSATILYEILLGKATLYAVLVSTLVVMAMVKRKNS |
| | (SEQ ID NO: 32) |
| mTCR 4734_1 alpha chain | GDKVKQSPSALSLQEGTNSALRCNFSIAATTVQWFLQ |
| (mouse TRAV4-3*03/ | NPRGSLINLFYLVPGTKENGRLKSAFDSKESYSTLHIR |
| TRAJ48*01) | DAQLEDSGTYFCAAHGYGNEKITFGAGTKLTIKPNIQ |
| (predicted sequence without | NPEPAVYQLKDPRSQDSTLCLFTDFDSQINVPKTMESGT |
| N-terminal signal peptide) | FITDKTVLDMKAMDSKSNGAIAWSNQTSFTCQDIFKETN |
| | ATYPSSDVPCDATLTEKSFETDMNLNFQNLSVMGLRILLL |
| | KVAGFNLLMTLRLWSS (SEQ ID NO: 33) |
| mTCR 4734 1 beta chain | GPKVLQIPSHQIIDMGQMVTLNCDPVSNHLYFYWYK |
| (mouse TRBV3*01/ | QILGQQMEFLVNFYNGKVMEKSKLFKDQFSVERPDG |
| TRBJ1-4*02/TRBD1*01) | SYFTLKIQPTALEDSAVYFCASSSRDRGRERLFFGHGT |
| (predicted sequence without | KLSVLEDLRNVTPPKVSLFEPSKAELANKQKATLVCLARG |
| N-terminal signal peptide) | FFPDHVELSWWVNGKEVHSGVSTDPQAYKESNYSYCLSS |
| | RLRVSATFWHNPRNHFRCQVQFHGLSEEDKWPEGSPKP |
| | VTQNISAEAWGRADCGITSASYQQGVLSATILYEILLGKAT |
| | LYAVLVSTLVVMAMVKRKNS (SEQ ID NO: 34) |
| mTCR 5489_4 alpha chain | MNMRPVTSSVLVLLLMLRRSNGDSVTQIEGLVTVTE |
| (mouse TRAV12-1*06/ | GLPVKLNCTYQTTYLTIAFFWYVQYLNEAPQVLLKSS |
| TRAJ26*01) | TDNKRTEHQGFHATLHKSSSSFHLQKSSAQLSDSALY |
| (with N-terminal signal | YCALSDRGNYAQGLTFGLGTRVSVFPYIQNPEPAVYQL |
| peptide) | KDPRSQDSTLCLFTDFDSQINVPKTMESGTFITDKTVLD |
| | MKAMDSKSNGAIAWSNQTSFTCQDIFKETNATYPSSDVP |
| | CDATLTEKSFETDMNLNFQNLSVMGLRILLLKVAGENLL |
| | MTLRLWSS (SEQ ID NO: 39) |
| mTCR 5489_4 beta chain | MGSIFLSCLAVCLLVAGPVDPKIIQKPKYLVAVTGSE |
| (mouse TRBV2*01/ | KILICEQYLGHNAMYWYRQSAKKPLEFMFSYSYQKL |
| TRBJ2-7*01/TRBD1*01) | MDNQTASSRFQPQSSKKNHLDLQITALKPDDSATYFC |
| (with N-terminal signal | ASSQEGSYEQYFGPGTRLTVLEDLRNVTPPKVSLFEPSK |
| peptide) | AEIANKQKATLVCLARGFFPDHVELSWWVNGKEVHSGVS |
| | TDPQAYKESNYSYCLSSRLRVSATFWHNPRNHFRCQVQF |
| | HGLSEEDKWPEGSPKPVTQNISAEAWGRADCGITSASYH |
| | QGVLSATILYEILLGKATLYAVLVSGLVLMAMVKKKNS |
| | (SEQ ID NO: 40) |
| mTCR 5489_4 alpha chain | GDSVTQIEGLVTVTEGLPVKLNCTYQTTYLTIAFFWY |
| (mouse TRAV12-1*06/ | VQYLNEAPQVLLKSSTDNKRTEHQGFHATLHKSSSSF |
| TRAJ26*01) | HLQKSSAQLSDSALYYCALSDRGNYAQGLTFGLGTR |
| (predicted sequence without | VSVFPYIQNPEPAVYQLKDPRSQDSTLCLFTDFDSQINVP |
| N-terminal signal peptide) | KTMESGTFITDKTVLDMKAMDSKSNGAIAWSNQTSFTCQ |
| | DIFKETNATYPSSDVPCDATLTEKSFETDMNLNFQNLSV |
| | MGLRILLLKVAGFNLLMTLRLWSS (SEQ ID NO: 41) |
| mTCR 5489_4 beta chain | DPKIIQKPKYLVAVTGSEKILICEQYLGHNAMYWYRQ |
| (mouse TRBV2*01/ | SAKKPLEFMFSYSYQKLMDNQTASSRFQPQSSKKNHL |
| TRBJ2-7*01/TRBD1*01) | DLQITALKPDDSATYFCASSQEGSYEQYFGPGTRLTVL |
| (predicted sequence without | EDLRNVTPPKVSLFEPSKAELANKQKATLVCLARGFFPDH |
| N-terminal signal peptide) | VELSWWVNGKEVHSGVSTDPQAYKESNYSYCLSSRLRVSA |
| | TFWHNPRNHFRCQVQFHGLSEEDKWPEGSPKPVTQNIS |
| | AEAWGRADCGITSASYHQGVLSATILYEILLGKATLYAVLV |
| | SGLVLMAMVKKKNS (SEQ ID NO: 42) |

### Example 2

This example demonstrates the preparation of retroviral vectors encoding the respective TCRs of Example 1.

The nucleotide sequences encoding the mTCR 4734_1 and mTCR 5489_4 alpha and beta chains of Table 4 were codon-optimized. Each codon-optimized mTCR 4734_1 and mTCR 5489_4 alpha and beta chain pair was independently cloned into MSGV1 retroviral vectors. A furin/SGSG/P2A linker having the amino acid sequence of RAKRSGSGATNFSLLKQAGDVEENPGP (SEQ ID NO: 43) was positioned between the alpha and beta chains. The configuration of each retroviral vector was as follows: amino terminus (N)-beta chain-linker-alpha chain-carboxyl terminus (C).

### Example 3

This example demonstrates that human T cells transduced with a retroviral vector encoding mTCR 4734_1 or mTCR 5489_4 recognize target cells that express both HLA-A3 and V600E BRAF.

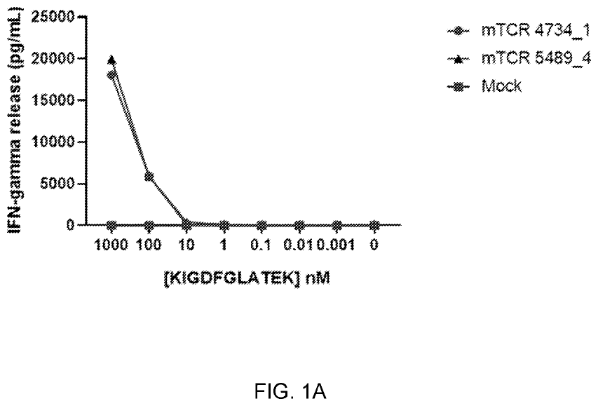

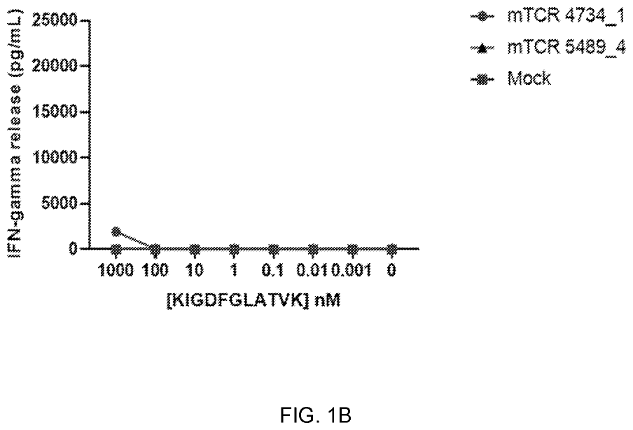

The retroviral constructs of Example 2 were used to produce retroviral particles. Human T cells were independently retrovirally transduced with the mTCR 4734_1 or mTCR 5489_4 retroviral particles. Transduced cells were co-cultured with HLA-A*03:01+ target cells that had been pulsed with either KIGDFGLATEK (mutated) (SEQ ID NO: 45) or KIGDFGLATVK (wild-type) (SEQ ID NO: 46) peptide. IFN-gamma release was measured by ELISA. The results are shown in FIGS. 1A and 1B.

The results showed that human T cells transduced with a retroviral vector encoding mTCR 4734_1 or mTCR 5489_4 recognized target cells that express both HLA-A3 and V600E BRAF.

### Example 4

This example demonstrates that human T cells transduced with a retroviral vector encoding mTCR 4734_1 or mTCR 5489_4 recognize endogenously processed V600E BRAF antigen on HLA-A*03:01+ target cells.

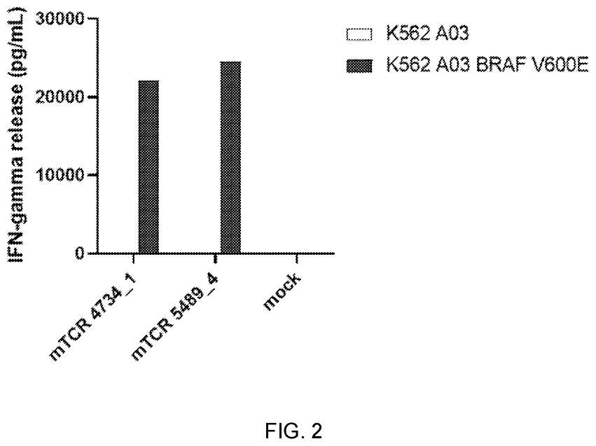

The retroviral constructs of Example 2 were used to produce retroviral particles. Human T cells were independently retrovirally transduced with the retroviral particles to express the V600E BRAF-specific TCRs. Human T cells transduced with an empty vector (mock) served as a control. Transduced cells were co-cultured with an HLA-A*03:01+ positive tumor cell line K562 that was either untransfected or transfected with V600E BRAF. Interferon-gamma secretion was measured. The results are shown in FIG. 2.

The results showed that human T cells transduced with a retroviral vector encoding mTCR 4734_1 or mTCR 5489_4 recognized endogenously processed V600E BRAF antigen on HLA-A*03:01+ target cells.

### Example 5

This example demonstrates that human T cells transduced with a retroviral vector encoding mTCR 4734_1 or mTCR 5489_4 recognize HLA-A*03:01+ melanoma cell lines with the V600E BRAF mutation.

The retroviral constructs of Example 2 were used to produce retroviral particles. Human T cells were independently retrovirally transduced with the retroviral particles to express the V600E BRAF-specific TCRs. Human T cells transduced with an empty vector (mock) served as a control.

Transduced cells (effector cells) were co-cultured with one of the following target tumor cell lines: Mel 2650, Mel 3333, Mel 3707, Mel 3709, Mel 3733, or Mel 3784. The target tumor cell lines were either (I) pulsed with KIGDFGLATEK (SEQ ID NO: 45) (positive control) or KIGDFGLATVK (SEQ ID NO: 46) (negative control) or (ii) not pulsed with any mutated or WT BRAF peptide. IFN-γ was measured. The results are shown in Tables 5-7. Tables 5-7 show the concentration of IFN-gamma (pg/mL) measured following the co-culture. In Tables 5-7, the “media” columns provide the results obtained following co-culture with target cells that were not pulsed with any mutated or WT BRAF peptide, and the columns labeled with the BRAF peptides indicate the results obtained following co-culture with target cells that were pulsed with the indicated BRAF peptide. All of the tumor cell lines in Tables 5-7 naturally express HLA-A*03:01 and BRAF V600E.

TABLE 5

| | | | 1 μM |
|---|---|---|---|
| | | 1 μM | KIGDFGLATEK |
| mTCR | | KIGDFGLATVK | (mutated) (SEQ ID NO: |
| 4374_1 | media | (WT) (SEQ ID NO: 46) | 45) |
| Mel 2650 | 0 | 288 | 8868 |
| Mel 3333 | 18 | 475 | 18344 |
| Mel 3707 | 0 | 34 | 11092 |
| Mel 3709 | 0 | 69 | 7926 |
| Mel 3733 | 18 | 210 | 11439 |
| Mel 3784 | 0 | 35 | 3143 |

TABLE 6

| | | | 1 μM |
|---|---|---|---|
| | | 1 μM | KIGDFGLATEK |
| mTCR | | KIGDFGLATVK | (mutated) (SEQ ID NO: |
| 5489_4 | media | (WT) (SEQ ID NO: 46) | 45) |
| Mel 2650 | 220 | 204 | 8440 |
| Mel 3333 | 54 | 41 | 19140 |
| Mel 3707 | 18 | 0 | 13308 |
| Mel 3709 | 153 | 119 | 6736 |
| Mel 3733 | 1870 | 684 | 12319 |
| Mel 3784 | 24 | 16 | 3141 |

TABLE 7

| | | | 1 μM KIGDFGLATEK |
|---|---|---|---|
| | | 1 μM KIGDFGLATVK | (SEQ ID NO: 45) |
| Mock | media | (SEQ ID NO: 46) (WT) | (mutated) |
| Mel 2650 | 32 | 18 | 29 |
| Mel 3333 | 36 | 16 | 23 |
| Mel 3707 | 0 | 0 | 0 |
| Mel 3709 | 0 | 0 | 0 |
| Mel 3733 | 45 | 16 | 20 |
| Mel 3784 | 0 | 0 | 0 |

As shown in Tables 5-7, human T cells transduced with a retroviral vector encoding mTCR 4734_1 or mTCR 5489_4 specifically recognized tumor cells that express both HLA-A3 and V600E BRAF.

### Example 6

This example demonstrates that human T cells transduced with a retroviral vector encoding mTCR 4734_1 or mTCR 5489_4 recognize an HLA-A*03:01+ melanoma cell line with the V600E BRAF mutation in an HLA class I-restricted manner.

The retroviral constructs of Example 2 were used to produce retroviral particles. Human T cells were independently retrovirally transduced with the retroviral particles to express the V600E BRAF-specific TCRs. Human T cells transduced with an empty vector (mock) served as a control.

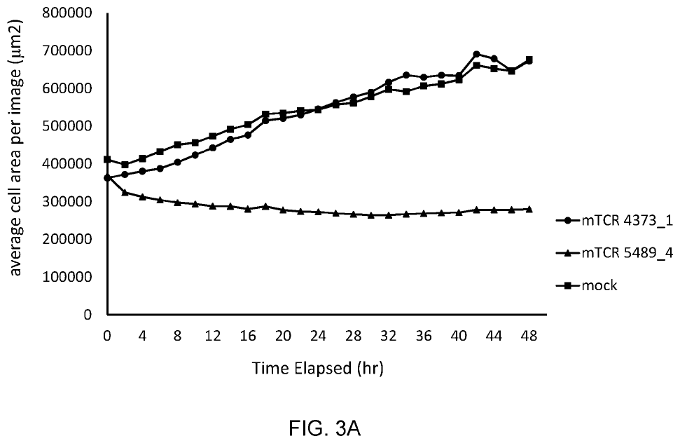

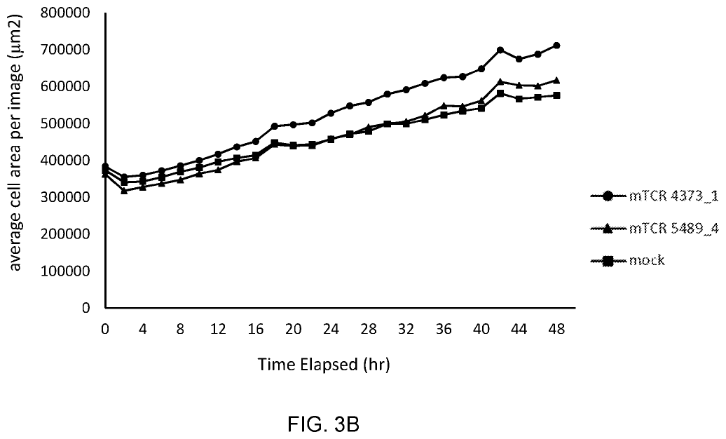

Transduced cells (effector cells) were co-cultured with tumor cell line mel 3733 (target cells) in the presence or absence of W6/32 antibodies. W6/32 is a mouse anti-HLA class I monoclonal antibody. Cytotoxicity was measured over the course of the 48 hour co-culture and evidenced by a reduction in the area covered by viable target cells using the INCUCYTE live-cell analysis system. The results are shown in FIGS. 3A-3B. The results showed that mTCR 4734_1 and mTCR 5489_4 recognized the target cells in an HLA class I-restricted manner.

### Example 7

This example demonstrates that human T cells transduced with a retroviral vector encoding mTCR 5489_4 recognize HLA-A*03:01+ melanoma cell lines with the V600E BRAF mutation.

The retroviral construct encoding the mTCR 5489_4 of Example 2 was used to produce retroviral particles. Peripheral blood lymphocytes (PBLs) from two healthy donors were retrovirally transduced with the retroviral particles to express mTCR 5489_4. PBLs transduced with an empty vector (mock) served as a control.

Transduced cells (effector cells) were co-cultured with a target tumor cell line either derived from a patient tumor (hTC) or from a xenograft (xTC), namely Mel 2650 hTC, Mel 2650 xTC, Mel 3707 hTC, Mel 3707 xTC, Mel 3709 hTC, Mel 3733 hTC, Mel 3733 XTC, Mel 3784 hTC, or Mel 3784 xTC, or with a target tumor organoid from a colorectal cancer patient (Crc 4487 org). IFN-γ was measured. The results are shown in Table 8. Table 8 shows the concentration of IFN-gamma (pg/mL) measured following the co-culture (“NT”=not tested). All of the tumor cell lines in Table 8 naturally express HLA-A*03:01 and BRAF V600E.

TABLE 8

| | Healthy Donor No. 1 PBL | | Healthy Donor No. 2 PBL | |
|---|---|---|---|---|
| | mTCR | Mock | mTCR | Mock |
| Targets | 5489_4 | TCR | 5489_4 | TCR |
| Mel 2650 hTC | 124 | 0 | NT | NT |
| Mel 2650 xTC | 201 | 0 | 458 | 0 |
| Mel 3707 hTC | 153 | 0 | 520 | 0 |
| Mel 3707 xTC | NT | NT | 755 | 0 |
| Mel 3709 hTC | 95 | 0 | NT | NT |
| Mel 3733 hTC | 119 | 0 | NT | NT |
| Mel 3733 xTC | NT | NT | 496 | 0 |
| Mel 3784 hTC | 155 | 0 | 407 | 0 |
| Mel 3784 xTC | NT | NT | 501 | 0 |
| Crc 4487 org | 290 | 24 | NT | NT |

As shown in Table 8, human PBLs transduced with a retroviral vector encoding mTCR 5489_4 specifically recognized tumor cells that express both HLA-A3 and V600E BRAF.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated or purified T cell receptor (TCR) having antigenic specificity for a human BRAF^{V600E} amino acid sequence, wherein the TCR comprises the amino acid sequences of:
(a) all of SEQ ID NO: 1-3;
(b) all of SEQ ID NO: 4-6;
(c) all of SEQ ID NO: 1-6;
(d) all of SEQ ID NO: 11-13;
(e) all of SEQ ID NO: 14-16; or
(f) all of SEQ ID NO: 11-16.

2-4. (canceled)

5. The TCR according to claim 1, comprising the amino acid sequences of:
(i) SEQ ID NO: 7,
(ii) SEQ ID NO: 8,
(iii) SEQ ID NO: 9,
(iv) SEQ ID NO: 10,
(v) both of SEQ ID NOs: 7 and 8,
(vi) both of SEQ ID NOs: 9 and 10,
(vii) SEQ ID NO: 17,
(viii) SEQ ID NO: 18,
(ix) SEQ ID NO: 19,
(x) SEQ ID NO: 20,
(xi) both of SEQ ID NOs: 17 and 18, or
(xii) both of SEQ ID NOs: 19 and 20.

6. The TCR of claim 1, further comprising:
(a) an α chain constant region comprising the amino acid sequence of SEQ ID NO: 21, wherein:
(i) X at position 47 of SEQ ID NO: 21 is Thr or Cys;
(ii) X at position 111 of SEQ ID NO: 21 is Ser, Ala, Val, Leu, Ile, Pro, Phe, Met, or Trp;
(iii) X at position 113 of SEQ ID NO: 21 is Met, Ala, Val, Leu, Ile, Pro, Phe, or Trp; and
(iv) X at position 114 of SEQ ID NO: 21 is Gly, Ala, Val, Leu, Ile, Pro, Phe, Met, or Trp;
(b) a β chain constant region comprising the amino acid sequence of SEQ ID NO: 22, wherein X at position 59 of SEQ ID NO: 22 is Ser or Cys;
(c) a β chain constant region comprising the amino acid sequence of SEQ ID NO: 23, wherein X at position 59 of SEQ ID NO: 23 is Ser or Cys;
(d) both (a) and (b); or
(e) both (a) and (c).

7. (canceled)

8. An isolated or purified polypeptide comprising a functional portion of the TCR of claim 1, wherein the functional portion comprises the amino acid sequences of:
(a) all of SEQ ID NO: 1-3;
(b) all of SEQ ID NO: 4-6;
(c) all of SEQ ID NO: 1-6;
(d) all of SEQ ID NO: 11-13;
(e) all of SEQ ID NO: 14-16; or
(f) all of SEQ ID NO: 11-16.

9. The isolated or purified polypeptide according to claim 8, wherein the functional portion comprises the amino acid sequence(s) of:
(i) SEQ ID NO: 7,
(ii) SEQ ID NO: 8,
(iii) SEQ ID NO: 9,
(iv) SEQ ID NO: 10,
(v) both of SEQ ID NOs: 7 and 8,
(vi) both of SEQ ID NOs: 9 and 10,
(vii) SEQ ID NO: 17,
(viii) SEQ ID NO: 18,
(ix) SEQ ID NO: 19,
(x) SEQ ID NO: 20,
(xi) both of SEQ ID NOs: 17 and 18, or
(xii) both of SEQ ID NOs: 19 and 20.

10. The isolated or purified polypeptide of claim 8, further comprising:
(a) an α chain constant region comprising the amino acid sequence of SEQ ID NO: 21, wherein:
(i) X at position 47 of SEQ ID NO: 21 is Thr or Cys;
(ii) X at position 111 of SEQ ID NO: 21 is Ser, Ala, Val, Leu, Ile, Pro, Phe, Met, or Trp;
(iii) X at position 113 of SEQ ID NO: 21 is Met, Ala, Val, Leu, Ile, Pro, Phe, or Trp; and
(iv) X at position 114 of SEQ ID NO: 21 is Gly, Ala, Val, Leu, Ile, Pro, Phe, Met, or Trp;
(b) a β chain constant region comprising the amino acid sequence of SEQ ID NO: 22, wherein X at position 59 of SEQ ID NO: 22 is Ser or Cys;
(c) a β chain constant region comprising the amino acid sequence of SEQ ID NO: 23, wherein X at position 59 of SEQ ID NO: 23 is Ser or Cys;
(d) both (a) and (b); or
(e) both (a) and (c).

11. (canceled)

12. An isolated or purified protein comprising at least one of the polypeptides of claim 8.

13. The isolated or purified protein according to claim 12, comprising:
(a) a first polypeptide chain comprising the amino acid sequences of SEQ ID NOs: 1-3;
(b) a second polypeptide chain comprising the amino acid sequences of SEQ ID NOs: 4-6;
(c) both (a) and (b);
(d) a first polypeptide chain comprising the amino acid sequences of SEQ ID NOS: 11-13;
(e) a second polypeptide chain comprising the amino acid sequences of SEQ ID NOs: 14-16; or
(f) both (d) and (e)

14. The isolated or purified protein according to claim 13, wherein:
(a) the first polypeptide chain comprises the amino acid sequence of SEQ ID NO: 7;
(b) the second polypeptide chain comprises the amino acid sequence of SEQ ID NO: 8;
(c) the first polypeptide chain comprises the amino acid sequence of SEQ ID NO: 9;
(d) the second polypeptide chain comprises the amino acid sequence of SEQ ID NO: 10;
(e) both (a) and (b);
(f) both (c) and (d);
(g) the first polypeptide chain comprises the amino acid sequence of SEQ ID NO: 17;
(h) the second polypeptide chain comprises the amino acid sequence of SEQ ID NO: 18;
(i) the first polypeptide chain comprises the amino acid sequence of SEQ ID NO: 19;
(j) the second polypeptide chain comprises the amino acid sequence of SEQ ID NO: 20;
(k) both (g) and (h); or
(l) both (i) and (j).

15. The isolated or purified protein of claim 13, wherein:
(a) the first polypeptide chain further comprises the amino acid sequence of SEQ ID NO: 21, wherein:
(i) X at position 47 of SEQ ID NO: 21 is Thr or Cys;
(ii) X at position 111 of SEQ ID NO: 21 is Ser, Ala, Val, Leu, Ile, Pro, Phe, Met, or Trp;
(iii) X at position 113 of SEQ ID NO: 21 is Met, Ala, Val, Leu, Ile, Pro, Phe, or Trp; and
(iv) X at position 114 of SEQ ID NO: 21 is Gly, Ala, Val, Leu, Ile, Pro, Phe, Met, or Trp;
(b) the second polypeptide chain further comprises the amino acid sequence of SEQ ID NO: 22, wherein X at position 59 of SEQ ID NO: 22 is Ser or Cys;
(c) the second polypeptide chain further comprises the amino acid sequence of SEQ ID NO: 23, wherein X at position 59 of SEQ ID NO: 23 is Ser or Cys;
(d) both (a) and (b); or
(e) both (a) and (c).

16. (canceled)

17. A bispecific engager TCR fusion protein comprising (i) the TCR according to claim 1 and (ii) an anti-CD3 effector.

18. An isolated or purified nucleic acid comprising a nucleotide sequence encoding the TCR according to claim 1 or a recombinant expression vector comprising the nucleic acid.

19. An isolated or purified nucleic acid or a recombinant expression vector comprising the nucleic acid, wherein the nucleic acid comprises, from 5′ to 3′, a first nucleotide sequence and a second nucleotide sequence, wherein the first and second nucleotide sequence, respectively, encode the amino sequences of SEQ ID NOs: 7 and 8; 8 and 7; 9 and 10; 10 and 9; 17 and 18; 18 and 17; 19 and 20; 20 and 19; 27 and 28; 28 and 27; 29 and 30; 30 and 29; 31 and 32; 32 and 31; 33 and 34; 34 and 33; 35 and 36; 36 and 35; 37 and 38; 38 and 37; 39 and 40; 40 and 39; 41 and 42; or 42 and 41.

20-25. (canceled)

26. A method of producing a host cell expressing a TCR that has antigenic specificity for the peptide of KIGDFGLATEK (SEQ ID NO: 45), the method comprising contacting a cell in vitro with the vector according to claim 18 under conditions that allow introduction of the vector into the cell.

27. An isolated or purified (i) host cell comprising the nucleic acid according to claim 18 or (ii) population of cells comprising the host cell.

28-30. (canceled)

31. A method of producing a T cell receptor (TCR), the method comprising culturing the host cell according to claim 27, so that the TCR is produced.

32. A pharmaceutical composition comprising (a) the population of cells according to claim 27 and (b) a pharmaceutically acceptable carrier.

33. A method of detecting the presence of cancer in mammal, the method comprising:
(a) contacting a sample comprising cells of the cancer with the TCR according to claim 1, thereby forming a complex; and
(b) detecting the complex,
wherein detection of the complex is indicative of the presence of cancer in the mammal.

34-37. (canceled)

38. A method of treating or preventing cancer in a mammal, the method comprising administering to the mammal the population of cells according to claim 27 in an amount effective to treat or prevent cancer in the mammal.

39. A method of inducing an immune response against cancer in a mammal, the method comprising administering to the mammal the population of cells according to claim 27 in an amount effective to induce the immune response against cancer in the mammal.


SEQUENCE LISTING (48 sequences)

SEQ ID NO: 1 - protein, 5 aa, Mus musculus
IAATT

SEQ ID NO: 2 - protein, 5 aa, Mus musculus
LVPGT

SEQ ID NO: 3 - protein, 11 aa, Mus musculus
AAHGYGNEKIT

SEQ ID NO: 4 - protein, 5 aa, Mus musculus
SNHLY

SEQ ID NO: 5 - protein, 6 aa, Mus musculus
FYNGKV

SEQ ID NO: 6 - protein, 13 aa, Mus musculus
ASSSRDRGRERLF

SEQ ID NO: 7 - protein, 110 aa, Mus musculus
GDKVKQSPSALSLQEGTNSALRCNFSIAATTVQWFLQNPRGSLINLFYLVPGTKENGRLKSAFDSKESYSTLHIRDAQLEDSGTYFCAAHGYGNEKITFGAGTKLTIKPN

SEQ ID NO: 8 - protein, 113 aa, Mus musculus
GPKVLQIPSHQIIDMGQMVTLNCDPVSNHLYFYWYKQILGQQMEFLVNFYNGKVMEKSKLFKDQFSVERPDGSYFTLKIQPTALEDSAVYFCASSSRDRGRERLFFGHGTKLS

SEQ ID NO: 9 - protein, 130 aa, Mus musculus
MQRNLGAVLGILWVQICWVSGDKVKQSPSALSLQEGTNSALRCNFSIAATTVQWFLQNPRGSLINLFYLVPGTKENGRLKSAFDSKESYSTLHIRDAQLEDSGTYFCAAHGYGNEKITFGAGTKLTIKPN

SEQ ID NO: 10 - protein, 132 aa, Mus musculus
MDIWLLGWIIFSFLEAGHTGPKVLQIPSHQIIDMGQMVTLNCDPVSNHLYFYWYKQILGQQMEFLVNFYNGKVMEKSKLFKDQFSVERPDGSYFTLKIQPTALEDSAVYFCASSSRDRGRERLFFGHGTKLS

SEQ ID NO: 11 - protein, 7 aa, Mus musculus
TTYLTIA

SEQ ID NO: 12 - protein, 7 aa, Mus musculus
SSTDNKR

SEQ ID NO: 13 - protein, 13 aa, Mus musculus
ALSDRGNYAQGLT

SEQ ID NO: 14 - protein, 5 aa, Mus musculus
LGHNA

SEQ ID NO: 15 - protein, 6 aa, Mus musculus
YSYQKL

SEQ ID NO: 16 - protein, 11 aa, Mus musculus
ASSQEGSYEQY

SEQ ID NO: 17 - protein, 116 aa, Mus musculus
GDSVTQIEGLVTVTEGLPVKLNCTYQTTYLTIAFFWYVQYLNEAPQVLLKSSTDNKRTEHQGFHATLHKSSSSFHLQKSSAQLSDSALYYCALSDRGNYAQGLTFGLGTRVSVFPY

SEQ ID NO: 18 - protein, 110 aa, Mus musculus
DPKIIQKPKYLVAVTGSEKILICEQYLGHNAMYWYRQSAKKPLEFMFSYSYQKLMDNQTASSRFQPQSSKKNHLDLQITALKPDDSATYFCASSQEGSYEQYFGPGTRLT

SEQ ID NO: 19 - protein, 137 aa, Mus musculus
MNMRPVTSSVLVLLLMLRRSNGDSVTQIEGLVTVTEGLPVKLNCTYQTTYLTIAFFWYVQYLNEAPQVLLKSSTDNKRTEHQGFHATLHKSSSSFHLQKSSAQLSDSALYYCALSDRGNYAQGLTFGLGTRVSVFPY

SEQ ID NO: 20 - protein, 129 aa, Mus musculus
MGSIFLSCLAVCLLVAGPVDPKIIQKPKYLVAVTGSEKILICEQYLGHNAMYWYRQSAKKPLEFMFSYSYQKLMDNQTASSRFQPQSSKKNHLDLQITALKPDDSATYFCASSQEGSYEQYFGPGTRLT

SEQ ID NO: 21 - protein, 136 aa, synthetic construct
  VARIANT 47: note = X at position 47 is T or C
  VARIANT 111: note = X at position 111 is S, A, V, L, I, P, F, M, or W
  VARIANT 113: note = X at position 113 is M, A, V, L, I, P, F, or W
  VARIANT 114: note = X at position 114 is G, A, V, L, I, P, F, M, or W
IQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 22 - protein, 175 aa, synthetic construct
  VARIANT 59: note = X at position 59 is S or C
VLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 23 - protein, 175 aa, synthetic construct
  VARIANT 59: note = X at position 59 is S or C
VLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 24 - protein, 136 aa, Mus musculus
IQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 25 - protein, 175 aa, Mus musculus
VLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 26 - protein, 175 aa, Mus musculus
VLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 27 - protein, 266 aa, synthetic construct
  VARIANT 241: note = X at position 241 is S, A, V, L, I, P, F, M, or W
  VARIANT 243: note = X at position 243 is M, A, V, L, I, P, F, or W
  VARIANT 244: note = X at position 244 is G, A, V, L, I, P, F, M, or W
  VARIANT 177: note = X at position 177 is T or C
MQRNLGAVLGILWVQICWVSGDKVKQSPSALSLQEGTNSALRCNFSIAATTVQWFLQNPRGSLINLFYLVPGTKENGRLKSAFDSKESYSTLHIRDAQLEDSGTYFCAAHGYGNEKITFGAGTKLTIKPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 28 - protein, 307 aa, synthetic construct
  VARIANT 191: note = X at position 191 is S or C
MDIWLLGWIIFSFLEAGHTGPKVLQIPSHQIIDMGQMVTLNCDPVSNHLYFYWYKQILGQQMEFLVNFYNGKVMEKSKLFKDQFSVERPDGSYFTLKIQPTALEDSAVYFCASSSRDRGRERLFFGHGTKLSVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 29 - protein, 246 aa, synthetic construct
  VARIANT 157: note = X at position 157 is T or C
  VARIANT 221: note = X at position 221 is S, A, V, L, I, P, F, M, or W
  VARIANT 223: note = X at position 223 is M, A, V, L, I, P, F, or W
  VARIANT 224: note = X at position 224 is G, A, V, L, I, P, F, M, or W
GDKVKQSPSALSLQEGTNSALRCNFSIAATTVQWFLQNPRGSLINLFYLVPGTKENGRLKSAFDSKESYSTLHIRDAQLEDSGTYFCAAHGYGNEKITFGAGTKLTIKPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 30 - protein, 288 aa, synthetic construct
  VARIANT 172: note = X at position 172 is S or C
GPKVLQIPSHQIIDMGQMVTLNCDPVSNHLYFYWYKQILGQQMEFLVNFYNGKVMEKSKLFKDQFSVERPDGSYFTLKIQPTALEDSAVYFCASSSRDRGRERLFFGHGTKLSVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 31 - protein, 266 aa, Mus musculus
MQRNLGAVLGILWVQICWVSGDKVKQSPSALSLQEGTNSALRCNFSIAATTVQWFLQNPRGSLINLFYLVPGTKENGRLKSAFDSKESYSTLHIRDAQLEDSGTYFCAAHGYGNEKITFGAGTKLTIKPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 32 - protein, 307 aa, Mus musculus
MDIWLLGWIIFSFLEAGHTGPKVLQIPSHQIIDMGQMVTLNCDPVSNHLYFYWYKQILGQQMEFLVNFYNGKVMEKSKLFKDQFSVERPDGSYFTLKIQPTALEDSAVYFCASSSRDRGRERLFFGHGTKLSVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 33 - protein, 246 aa, Mus musculus
GDKVKQSPSALSLQEGTNSALRCNFSIAATTVQWFLQNPRGSLINLFYLVPGTKENGRLKSAFDSKESYSTLHIRDAQLEDSGTYFCAAHGYGNEKITFGAGTKLTIKPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 34 - protein, 288 aa, Mus musculus
GPKVLQIPSHQIIDMGQMVTLNCDPVSNHLYFYWYKQILGQQMEFLVNFYNGKVMEKSKLFKDQFSVERPDGSYFTLKIQPTALEDSAVYFCASSSRDRGRERLFFGHGTKLSVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 35 - protein, 273 aa, synthetic construct
  VARIANT 184: note = X at position 184 is T or C
  VARIANT 248: note = X at position 248 is S, A, V, L, I, P, F, M, or W
  VARIANT 251: note = X at position 251 is G, A, V, L, I, P, F, M, or W
  VARIANT 250: note = X at position 250 is M, A, V, L, I, P, F, or W
MNMRPVTSSVLVLLLMLRRSNGDSVTQIEGLVTVTEGLPVKLNCTYQTTYLTIAFFWYVQYLNEAPQVLLKSSTDNKRTEHQGFHATLHKSSSSFHLQKSSAQLSDSALYYCALSDRGNYAQGLTFGLGTRVSVFPYIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 36 - protein, 304 aa, synthetic construct
  VARIANT 188: note = X at position 188 is S or C
MGSIFLSCLAVCLLVAGPVDPKIIQKPKYLVAVTGSEKILICEQYLGHNAMYWYRQSAKKPLEFMFSYSYQKLMDNQTASSRFQPQSSKKNHLDLQITALKPDDSATYFCASSQEGSYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 37 - protein, 252 aa, synthetic construct
  VARIANT 163: note = X at position 163 is T or C
  VARIANT 227: note = X at position 227 is S, A, V, L, I, P, F, M, or W
  VARIANT 229: note = X at position 229 is M, A, V, L, I, P, F, or W
  VARIANT 230: note = X at position 230 is G, A, V, L, I, P, F, M, or W
GDSVTQIEGLVTVTEGLPVKLNCTYQTTYLTIAFFWYVQYLNEAPQVLLKSSTDNKRTEHQGFHATLHKSSSSFHLQKSSAQLSDSALYYCALSDRGNYAQGLTFGLGTRVSVFPYIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 38 - protein, 285 aa, synthetic construct
  VARIANT 169: note = X at position 169 is S or C
DPKIIQKPKYLVAVTGSEKILICEQYLGHNAMYWYRQSAKKPLEFMFSYSYQKLMDNQTASSRFQPQSSKKNHLDLQITALKPDDSATYFCASSQEGSYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 39 - protein, 273 aa, Mus musculus
MNMRPVTSSVLVLLLMLRRSNGDSVTQIEGLVTVTEGLPVKLNCTYQTTYLTIAFFWYVQYLNEAPQVLLKSSTDNKRTEHQGFHATLHKSSSSFHLQKSSAQLSDSALYYCALSDRGNYAQGLTFGLGTRVSVFPYIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 40 - protein, 304 aa, Mus musculus
MGSIFLSCLAVCLLVAGPVDPKIIQKPKYLVAVTGSEKILICEQYLGHNAMYWYRQSAKKPLEFMFSYSYQKLMDNQTASSRFQPQSSKKNHLDLQITALKPDDSATYFCASSQEGSYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 41 - protein, 252 aa, Mus musculus
GDSVTQIEGLVTVTEGLPVKLNCTYQTTYLTIAFFWYVQYLNEAPQVLLKSSTDNKRTEHQGFHATLHKSSSSFHLQKSSAQLSDSALYYCALSDRGNYAQGLTFGLGTRVSVFPYIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 42 - protein, 285 aa, Mus musculus
DPKIIQKPKYLVAVTGSEKILICEQYLGHNAMYWYRQSAKKPLEFMFSYSYQKLMDNQTASSRFQPQSSKKNHLDLQITALKPDDSATYFCASSQEGSYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 43 - protein, 27 aa, synthetic construct
RAKRSGSGATNFSLLKQAGDVEENPGP

SEQ ID NO: 44 - protein, 13 aa, Hepatitis B virus
TPPAYRPPNAPIL

SEQ ID NO: 45 - protein, 11 aa, Homo sapiens
KIGDFGLATEK

SEQ ID NO: 46 - protein, 11 aa, Homo sapiens
KIGDFGLATVK

SEQ ID NO: 47 - protein, 766 aa, Homo sapiens
MAALSGGGGGGAEPGQALFNGDMEPEAGAGAGAAASSAADPAIPEEVWNIKQMIKLTQEHIEALLDKFGGEHNPPSIYLEAYEEYTSKLDALQQREQQLLESLGNGTDFSVSSSASMDTVTSSSSSSLSVLPSSLSVFQNPTDVARSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETALTSGSSPSAPASDSIGPQILTSPSPSKSIPIPQPFRPADEDHRNQFGQRDRSSSAPNVHINTIEPVNIDDLIRDQGFRGDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATEKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

SEQ ID NO: 48 - protein, 766 aa, Homo sapiens [sequence omitted]